(19) United States
(12) Patent Application Publication
(10) Pub. No.: US 2012/0156150 A1
(43) Pub. Date: Jun. 21, 2012

(54) Composition for Improving Skin Condition Comprising Extract from Black Tea as Active Ingredient

(75) Inventors: Young Chul Kim, Daegu (KR); So Young Choi, Daegu (KR); Kyung Ok Lee, Daejeon (KR); Teresa Jeon, Daegu (KR)

(73) Assignee: Keimyung University Industry Academic Cooperation Foundation, Daegu (KR)

(21) Appl. No.: 13/277,538

(22) Filed: Oct. 20, 2011

(30) Foreign Application Priority Data
Dec. 17, 2010 (KR) 10-2010-0129616

(51) Int. Cl. A61K 8/97; A61Q 19/00; A61Q 19/02

(52) U.S. Cl. 424/62; 424/729

(57) ABSTRACT

Disclosed is a composition for whitening skin or improving skin wrinkles comprising black tea extract as an active ingredient. In a cell experiment for Melan-a cells and an animal experiment using the brown guinea pig model with artificial tanning spots induced by UVB, the black tea extract of the present disclosure greatly inhibited melanin synthesis. Also, in an experiment on SKH-1 hairless mouse, it remarkably inhibited wrinkle formation. Accordingly, the composition of the present disclosure can be developed as a skin whitening agent, an anti-wrinkle agent or a medicine for treating diseases associated with melanin hyperpigmentation, e.g. liver spots, freckles, senile spots or solar lentigo.

### CROSS-REFERENCE TO RELATED APPLICATIONS

This application claims priority under 35 U.S.C. §119 to Korean Patent Application No. 10-2010-0129616, filed on Dec. 17, 2010, in the Korean Intellectual Property Office, the disclosure of which is incorporated herein by reference in its entirety.

### TECHNICAL FIELD

The present disclosure relates to a composition for improving skin condition comprising black tea extract as an active ingredient, more particularly to a composition for whitening skin or improving skin wrinkles comprising black tea extract as an active ingredient.

### BACKGROUND

Increased melanin production and accumulation may induce various skin diseases including acquired hyperpigmentation such as melasma, postinflammatory melanoderma, solar lentigo, etc. (Cullen 1998; Urabe et al. 1998). Hyperpigmentation of the epithelium and skin may be caused by increased number of melanocytes or increased activity of melanin-producing enzymes (Ortonne and Nordlund 1998). Ultraviolet rays, chronic inflammation and skin friction as well as abnormal secretion of α-melanocyte stimulating hormones (α-MSH) may also cause such diseases (Im et al. 2002; Kang et al. 2002). Since acquired hyperpigmentation frequently occurs on the externally exposed parts, it has social psychological and cosmetic impacts and a lot of researches and efforts have been made to develop depigmenting agents. Melanogenesis occurs predominantly in lysosome-like structures known as melanosomes of melanocytes in the basal layer of the epidermis (Hearing 2005). The most common forms of melanin, black/brown eumelanin and yellow/red pheomelanin are derived from a common tyrosinase-dependent pathway with the same precursor, tyrosine. Three major melanosome-associated enzymes of the tyrosinase gene family are involved in melanin biosynthesis. Tyrosinase is the critical enzyme catalyzing the following two important reactions in melanin synthesis: hydroxylation of tyrosine into 3,4-dihydroxyphenylalanine (DOPA) and oxidation of DOPA into dopaquinone (Tripathi et al. 1992). Dopaquinone is spontaneously converted to dopachrome. Tyrosinase-related protein-2 (TRP-2)/dopachrome tautomerase (DCT) catalyzes the conversion of dopachrome to 5,6-dihydroxyindole-2-carboxylic acid (DHICA). Tyrosinase-related protein-1 (TRP-1; DHICA oxidase) catalyzes the oxidation of DHICA into indole-5,6-quinone-2-carboxylic acid. These two closely related structures, TRP-2/DCT and TRP-1, form unstable quinones that undergo further polymerization, finally yielding melanin (Mallick et al. 2005). Some depigmenting agents control skin pigmentation by inhibiting transcription and activation of tyrosinase as well as other melanin-producing enzymes related thereto—TRP-1, TRP-2 and/or peroxidase. Although known inhibitors such as hydroquinone, kojic acid and arbutin have been used to treat skin hyperpigmentation, it was recently found out that they are not safe for human health. Hydroquinone (HQ) inhibits RNA and DNA synthesis by generating quinone and reactive oxygen species, which cause damage to membrane lipids and membrane-bound proteins, and decreases the function of melanosome and tyrosinase activity up to 90% by interacting with copper at the active site of the enzyme (Briganti et al. 2003). In spite of its relatively high melanocyte-specific toxicity that improves hyperpigmentation in 14-70% of cases and its general safety, there are side effects of HQ such as skin irritation, contact dermatitis and (rarely) ochronosis, a blackish hyperpigmentation very resistant to treatment (Ortonne and Passeron 2005).

Arbutin, a natural β-glycoside of HQ isolated from the fruit of California buckeye (Aesculus californica), is a very effective tyrosinase inhibitor without affecting mRNA expression (Hori et al. 2004). It also inhibits the maturation of melanosome. Higher concentrations are more efficacious than lower concentrations, but a paradoxical pigment darkening may occur because of postinflammatory hyperpigmentation (Draelos 2007). Kojic acid, a fungal metabolic product from Aspergillus and Penicillum species, is commonly used in Asia as a whitening agent and a diet supplement. It acts as a tyrosinase inhibitor by chelating copper at the active site of the enzyme (Battaini et al. 2000). Although it is effective as a hypopigmenting agent, especially in combination with other substances, it has a high sensitizing potential and can cause contact dermatitis (Lim 1999). Many researches are carried out to search and develop plant-derived compounds exhibiting skin whitening effect without causing side effects in human. Teas are one of the most commonly consumed drinks over the world. Polyphenols are naturally-occurring compounds found in fresh tea leaves. The major four polyphenols found in fresh tea leaves are epigallocatechin gallate (EGCG), epigallocatechin, epicatechin gallate and epicatechin, with EGCG being the most abundant. The four catechins can compose up to 30% of the dry weight of a freshly picked leaf (Graham 1992). In order to prepare green tea, harvested fresh tea leaves are steamed or pan-dried at high temperature to minimize oxidation of natural catechins. In contrast, black tea is prepared by facilitating oxidation and polymerization of polyphenols by curling or crushing fresh tea leaves during fermentation. As a result, other polyphenols such as thearubigins and theaflavins which give the characteristic reddish brown color and bitter taste of black tea are produced (Sun et al. 2006). Theaflavins and green tea flavonols are known to have antioxidative activities because of their radical scavenging and metal chelating abilities (Serafini et al. 1996).

Skin aging is a process whereby structural integrity and physiological function are lost gradually due to extrinsic and intrinsic factors (Farage et al. 2008). Various and numerous clinical changes of the skin are induced, including wrinkling caused by UV radiation, sunburn, immunosuppression, cancer and premature skin aging (photoaging). Photodamage to connective tissues of the skin is the main cause of aging characteristic of the photodamaged skin (Fisher et al. 1997). When the skin is exposed to UV, reactive oxygen species (ROS) are produced in large quantities. The reactive oxygen species include not only non-radical species such as hydrogen peroxide but also free radicals such as superoxides, hydroxyl radical and peroxy radical (Cerutti 1991). In vivo, some of the reactive oxygen species play positive roles such as energy production, phagocytosis, control of cell growth and cell signaling. On the other hand, they cause oxidative damages to DNA, proteins, fatty acids and sugars. Such damages result in some harmful effects as follows: disturbance in cellular metabolism, morphological and ultrastructural changes, attack via regulatory pathway, and modification of differentiation, proliferation and apoptosis of skin cells (Svobodova et al. 2003; Zhaorigetu et al. 2003). Although the reactive oxygen species are constantly produced by keratinocytes and fibroblasts, they are promptly removed by non-enzymatic and enzymatic antioxidant substances. This defensive mechanism involves antioxidant enzymes such as superoxide dismutase, catalase and glutathione peroxidase as well as small molecules such as glutathione and vitamin C and E (Fridovich 1999).

Photodamage to the skin induces increased epidermal thickness and modification of biological and mechanical properties of the skin, ultimately leading to wrinkling. This process is related with the qualitative and/or quantitative change in the dermal extracellular matrix (ECM) components, including accumulation of the major dermal components collagen and elastin fibers and immunohistochemical changes (Chaquer et al. 1995). As skin ages, fibroblasts reduce in number and their function degrades, leading to reduced synthesis of extracellular matrix proteins such as collagen fibers and elastin fibers, decreased water content in skin cells, and structural change of the stratum corneum (SC). Exposure to UVB results in damage to the skin barrier through oxidation of the stratum corneum, leading to reduced water-holding capacity of the skin. UV radiation down-regulates collagen synthesis and induces expression of matrix metalloproteinases (MMPs) (Brennan et al. 2003; Brenneisen et al. 2002). MMPs are family members of zinc-dependent endoproteases having a broad substrate specificity and they can degrade all extracellular matrices (Kim et al. 2006). When human skin is acutely exposed to UV radiation in vivo, synthesis of several MMPs involved in breakdown of collagen such as MMP-1 (interstitial collagenase), MMP-3 (stromelysin-1) and MMP-9 (92-kdgelatinase B) is up-regulated (Honda et al. 2008). Exposure to UV radiation results in various changes related with immunosuppression in the epithelium. UV radiation may result in decreased density of epithelial Langerhans cells (LCs) and dendritic epithelial T cells. Also, it induces penetration of macrophages, granulocytes and T cells into the epithelium (Sluyter and Halliday 2000).

Retinoids are known to induce proliferation of keratinocytes and fibroblasts, promote collagen synthesis, and reduce expression of MMPs in skin (Varani et al. 1991). The retinoid family includes vitamin A (retinol), synthetic derivatives thereof and natural retinol derivatives such as retinaldehyde, retinoic acid and retinyl esters (Antille et al. 2004).

Various natural and synthetic retinoids have been developed for treatment of skin aging and some of them are known to have histological and clinical improving effects. However, most of those researches were on patients with photoaged skin. Although retinoids show therapeutic effect in skin aging, their use has been restricted to treatment of skin irritations such as burns, scaling or dermatitis (Mukherjee et al. 2006).

Plant extracts have been extensively used for topical application for treatment of wounds, aging and diseases (Hsu 2005). The antioxidant activity of herbal phenolic ingredients such as phenolic acids and flavonoids has gained attentions. Tea (Camellia sinensis L.) leaves contain a large quantity of flavonoids and polyphenols including catechins [(−)-epigallocatechin 3-gallate (EGCG), (−)-epicatechin 3-gallate (ECG), (−)-epigallocatechin (EGC) and (−)-epicatechin (EC)], and theaflavins and thearubigens are produced by oxidation during the preparation of black tea (Yen and Chen 1995; Ho et al. 1992). The epicatechin derivatives, commonly called ‘polyphenols’, are the active ingredients of green tea and are known to have antioxidant, anti-inflammatory and anticancer activities (Katiyar et al. 2001). EGCG is known to suppress skin cancer caused by UV radiation or carcinogens and reduce degradation of extracellular matrix. Also, green tea polyphenols are known to exert skin anti-aging effects by promoting proliferation of keratinocytes (Wang et al. 1991; Lee et al. 2005; Hsu et al. 2003). White tea is the least processed tea and is known to contain high concentrations of polyphenols and have whitening effect. Black tea is known to reduce the generation of sunburn cells induced by UV radiation (Record and Dreost 1998), have skin cancer-inhibiting and anti-inflammatory effects (Ratansooriya and Fernancho 2009) and have antioxidative and antibacterial activities (Bancirova 2010). However, the skin aging-inhibiting effect of black tea is not known as yet.

Throughout the specification, a number of publications and patent documents are referred to and cited. The disclosure of the cited publications and patent documents is incorporated herein by reference in its entirety to more clearly describe the state of the related art and the present disclosure.

### SUMMARY

The inventors of the present disclosure have studied to develop substances capable of improving skin condition from natural sources. As a result, they have demonstrated through experiments that black tea extract provides an excellent skin whitening effect by inhibiting melanin synthesis and also provides a wrinkle improving effect comparable to that of the existing wrinkle improving agent, retinoic acid.

The present disclosure is directed to providing a composition for whitening skin containing black tea extract as an active ingredient.

The present disclosure is also directed to providing a composition for improving wrinkles containing black tea extract as an active ingredient.

Other features and aspects will be apparent from the following detailed description, the drawings, and the claims.

### BRIEF DESCRIPTION OF THE DRAWINGS

The application file contains a drawing executed in color (FIG. 11). Copies of this patent or patent application with the color drawing will be provided by the Office upon request and payment of the necessary fee.

The above and other objects, features and advantages of the present disclosure will become apparent from the following description of certain exemplary embodiments given in conjunction with the accompanying drawings, in which:

FIG. 1 shows a result of measuring total polyphenol contents of a sample of the present disclosure;

FIG. 2 shows a result of measuring total flavonoid contents of a sample of the present disclosure;

FIG. 3 shows a result of measuring the electron donating ability of a sample of the present disclosure;

FIG. 4 shows a result of measuring the effect of a sample of the present disclosure on viability of Melan-a cells;

FIG. 5 shows a result of observing the morphology of Melan-a cells treated with a sample of the present disclosure [X200; panel A: untreated cells, panel B: kojic acid (12.5 μg/mL)-treated cells, panel C: EGCG (12.5 μg/mL)-treated cells, panel D: arbutin (12.5 μg/mL)-treated cells, panel E: green tea (12.5 μg/mL)-treated cells, panel F: white tea (12.5 μg/mL)-treated cells, panel G: black tea (12.5 μg/mL)-treated cells, panel H: black tea (6.25 μg/mL)-treated cells, panel I: black tea (3.125 μg/mL)-treated cells];

FIG. 6 shows a result of measuring the melanin synthesis inhibiting effect of a sample of the present disclosure for Melan-a cells;

FIG. 7 shows a result of measuring the inhibitory effect of a sample of the present disclosure against tyrosinase activity of Melan-a cells in cells 60 minutes after treatment with the sample;

FIG. 8 shows a result of measuring the inhibitory effect of a sample of the present disclosure against tyrosinase activity of Melan-a cells in cell extract 60 minutes after treatment with the sample;

FIG. 9 shows a result of measuring the effect of a sample of the present disclosure on the expression of tyrosinase in Melan-a cells;

FIG. 10 shows a result of measuring the effect of a sample of the present disclosure on the expression of mRNA of tyrosinase, TRP-1 and TRP-2 in Melan-a cells;

FIG. 11 shows a result of observing depigmenting effect after applying a sample of the present disclosure to the skin of a brown guinea pig for 4 weeks (panel C: saline-treated group, panel VC: vehicle-treated group, panel PC: 2% hydroquinone-treated group, panel E1: 2% EGCG-treated group, panel E2: 2% green tea-treated group, panel E3: 2% white tea-treated group, panel E4: 2% black tea-treated group);

FIG. 12 shows a result of artificially forming sunburn spots on the skin of a brown guinea pig and measuring the melanin index after applying a sample of the present disclosure for 4 weeks;

FIG. 13 shows a result of applying a sample of the present disclosure to the skin of a brown guinea pig for 4 weeks and histologically observing the skin [H&E stain, X200; panel A: saline-treated group (group C), panel B: vehicle-treated group (group VC), panel C: 2% black tea-treated group (group E4), panel D: 2% EGCG-treated group (group E1), panel E: 2% green tea-treated group (group E2), panel F: 2% hydroquinone-treated group (group PC), panel G: 2% white tea-treated group (group E3), panel H: non-treated group (normal group)];

FIG. 14 shows a result of applying a sample of the present disclosure to the skin of a brown guinea pig for 4 weeks and observing melanin pigmentation [F-M stain, X400; panel A: saline-treated group (group C), panel B: vehicle-treated group (group VC), panel C: 2% black tea-treated group (group E4), panel D: 2% EGCG-treated group (group E1), panel E: 2% green tea-treated group (group E2), panel F: 2% hydroquinone-treated group (group PC), panel G: 2% white tea-treated group (group E3), panel H: non-treated group (normal group)];

FIG. 15 shows a result of applying a sample of the present disclosure to the skin of a brown guinea pig for 4 weeks and histologically observing S-100(+) melanocytes [immunohistochemical S-100 stain, X400; panel A: saline-treated group (group C), panel B: vehicle-treated group (group VC), panel C: 2% black tea-treated group (group E4), panel D: 2% EGCG-treated group (group E1), panel E: 2% green tea-treated group (group E2), panel F: 2% hydroquinone-treated group (group PC), panel G: 2% white tea-treated group (group E3), panel H: non-treated group (normal group)];

FIG. 16 shows a result of applying a sample of the present disclosure to the skin of a brown guinea pig for 4 weeks and histologically observing gp 100(+) melanocytes [immunohistochemical HMB-45 stain, X400; panel A: saline-treated group (group C), panel B: vehicle-treated group (group VC), panel C: 2% black tea-treated group (group E4), panel D: 2% EGCG-treated group (group E1), panel E: 2% green tea-treated group (group E2), panel F: 2% hydroquinone-treated group (group PC), panel G: 2% white tea-treated group (group E3), panel H: non-treated group (normal group)];

FIG. 17 shows a result of measuring total polyphenol and flavonoid contents of green tea extract (GT) and black tea extract (BT);

FIG. 18 shows a result of measuring the electron donating ability of green tea extract (GT) and black tea extract (BT);

FIG. 19 shows a result of applying a sample to an SKH-1 hairless mouse for 6 weeks and comparing total wrinkle area;

FIG. 20 shows a result of applying a sample to an SKH-1 hairless mouse for 6 weeks and comparing the replica image of the skin wrinkle area (panel A: normal group, panel B: control group, panel C, 0.01% retinoic acid-treated group, panel D: 2% green tea extract-treated group, panel E: 2% white tea extract-treated group, panel F: 2% black tea extract-treated group);

FIG. 21 shows a result of applying a sample to an SKH-1 hairless mouse for 6 weeks and histologically observing the skin tissue [(A): H&E stain, X200, (B): Masson's trichrome stain, X200, (C): Verhoeff's stain, X200, (D): toluidine blue stain, X200. N: normal group, C: control group, RA 0.01%: 0.01% retinoic acid-treated group, GT 2%: 2% green tea extract-treated group, WT 2%: 2% white tea extract-treated group, BT 2%: 2% black tea extract-treated group];

FIG. 22 shows a result of applying a sample to an SKH-1 hairless mouse for 6 weeks and comparing the expression of MMP-3 mRNA;

FIG. 23 shows a result of applying a sample to an SKH-1 hairless mouse for 6 weeks and comparing the activity of MMP-2 and MMP-9 proteins;

FIG. 24 shows a result of measuring the body weight of an SKH-1 hairless mouse during the test period.

### DETAILED DESCRIPTION OF EMBODIMENTS

The advantages, features and aspects of the present disclosure will become apparent, from the following description of the embodiments with reference to the accompanying drawings, which is set forth hereinafter. The present disclosure may, however, be embodied in different forms and should not be construed as limited to the embodiments set forth herein. Rather, these embodiments are provided so that this disclosure will be thorough and complete, and will fully convey the scope of the present disclosure to those skilled in the art. The terminology used herein is for the purpose of describing particular embodiments only and is not intended to be limiting of the example embodiments. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

Hereinafter, exemplary embodiments will be described in detail with reference to the accompanying drawings.

In a general aspect, the present disclosure provides a composition for whitening skin or improving wrinkles comprising black tea extract as an active ingredient.

In another general aspect, the present disclosure provides a pharmaceutical composition comprising black tea extract as an active ingredient for treating or preventing melanin hyperpigmentation diseases.

In another general aspect, the present disclosure provides a method of skin treatment, which comprises topically applying a topical composition comprising black tea extract on one or more areas of the skin of a person, thereby achieving reduction of pigmentation spots or degree of wrinkles.

The black tea extract of the present disclosure may be isolated according to a method commonly employed in the art to obtain extracts from natural products, i.e. under common temperature and pressure conditions using a commonly used solvent.

The extraction solvent for obtaining the black tea extract of the present disclosure may be a solvent that can be usually used in an extraction process. For example, a solvent selected from the group consisting of water, absolute or aqueous lower alcohol containing 1-4 carbons, acetone, ethyl acetate, butyl acetate and 1,3-butylene glycol may be used for extraction.

In an exemplary embodiment of the present disclosure, the black tea extract may be an extract obtained using water as a solvent. Most specifically, the black tea may be a hot water extract.

In an exemplary embodiment of the present disclosure, the black tea extract is included in an amount of 0.0001-10 wt % based on the total weight of the composition.

As demonstrated in the following examples, the black tea extract of the present disclosure greatly inhibits melanin synthesis in Melan-a cells in an animal experiment using a brown guinea pig model with artificial tanning spots induced by UVB and remarkably improves wrinkles in an experiment using an SKH-1 hairless mouse.

The composition of the present disclosure may be provided in the form of a cosmetic composition for whitening skin or improving wrinkles.

The cosmetic composition of the present disclosure may be prepared into any formulation common in the art. For example, it may be formulated into solution, suspension, emulsion, paste, gel, cream, lotion, powder, soap, surfactant-containing cleanser, oil, powder foundation, emulsion foundation, wax foundation, spray, etc., but without being limited thereto. More specifically, it may be formulated into emollient lotion, nourishing lotion, nourishing cream, massage cream, essence, eye cream, cleansing cream, cleansing foam, cleansing water, pack, spray or powder.

When the formulation of the present disclosure is in the form of paste, cream or gel, animal oil, plant oil, wax, paraffin, starch, tragacanth, cellulose derivatives, polyethylene glycol, silicone, bentonite, silica, talc, zinc oxide, etc. may be used as a carrier.

When the formulation of the present disclosure is in the form of powder or spray, lactose, talc, silica, aluminum hydroxide, calcium silicate or polyamide powder may be used as a carrier. Especially, when it is in the form of spray, the formulation may further comprise a propellant such as chlorofluorohydrocarbon, propane/butane or dimethyl ether.

When the formulation of the present disclosure is in the form of solution or emulsion, a solvent, solubilizer or emulsifier may be used as a carrier. For example, water, ethanol, isopropanol, ethyl carbonate, ethyl acetate, benzyl alcohol, benzyl benzoate, propylene glycol, 1,3-butylglycol oil, glycerol aliphatic ester, polyethylene glycol or fatty acid ester of sorbitan may be used.

When the formulation of the present disclosure is in the form of suspension, a liquid diluent such as water, ethanol or propylene glycol, a suspending agent such as ethoxylated isostearyl alcohol, polyoxyethylene sorbitol ester and polyoxyethylene sorbitan ester, microcrystalline cellulose, aluminum metahydroxide, bentonite, agar, tragacanth, etc. may be used as a carrier.

When the formulation of the present disclosure is in the form of surfactant-containing cleanser, aliphatic alcohol sulfate, aliphatic alcohol ether sulfate, sulfosuccinic monoester, isethionate, imidazolinium derivatives, methyl taurate, sarcosinate, fatty acid amide ether sulfate, alkyl amidobetaine, aliphatic alcohol, fatty acid glyceride, fatty acid diethanolamide, vegetable oil, lanolin derivatives, ethoxylated glycerol fatty acid ester, etc. may be used as a carrier.

The cosmetic composition of the present disclosure may further comprise, in addition to the black tea extract as the active ingredient as well as the carrier, other components commonly included in the cosmetic composition. For example, common adjuvants such as antioxidant, stabilizer, solubilizer, vitamin, pigment and fragrance may be included.

In an exemplary embodiment of the present disclosure, the cosmetic composition of the present disclosure may be in the form selected from the group consisting of solution, suspension, emulsion, paste, gel, cream, lotion, powder, soap, surfactant-containing cleanser, oil, powder foundation, emulsion foundation, wax foundation and spray.

In another general aspect, the present disclosure provides a pharmaceutical composition for treating or preventing melanin hyperpigmentation diseases comprising the black tea extract as an active ingredient.

As used herein, the term “melanin hyperpigmentation” refers to the darkening of a particular area of skin or nails as compared to other areas caused by excessive increase in melanin. Specifically, the melanin hyperpigmentation diseases may include liver spots, freckles, senile spots or solar lentigines, but are not limited thereto.

The pharmaceutical composition of the present disclosure may comprise a pharmaceutically acceptable carrier in addition to the active ingredient. The carrier may be, for example, lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, mineral oil, etc., but is not limited thereto. The pharmaceutical composition of the present disclosure may further include, in addition to the above-described components, a lubricant, a wetting agent, a sweetener, a fragrance, an emulsifier, a suspending agent, a preservative, or the like. Suitable pharmaceutically acceptable carriers and formulations are described in detail in Remington's Pharmaceutical Sciences (19th ed., 1995).

An appropriate dosage of the pharmaceutical composition of the present disclosure may be determined variously depending on such factors as preparation method, administration method, age, body weight and sex of the patient, pathological condition, diet, administration time, administration route, excretion rate or response sensitivity. Specifically, an oral dosage of the pharmaceutical composition of the present disclosure for an adult may be 0.0001-100 mg/kg (body weight) per day.

The pharmaceutical composition of the present disclosure may be administered orally or parenterally. When administered parenterally, it may be administered topically, intravenously, subcutaneously, intramuscularly, intraabdominally or transdermally.

Specifically, considering that the pharmaceutical composition of the present disclosure is used for treatment or prevention of melanin hyperpigmentation-related diseases, the present disclosure composition may be administered by topically applying to the skin.

The concentration of the active ingredient included in the composition of the present disclosure may be determined considering purpose of treatment, patient's condition, required period, or the like, and is not particularly limited.

The pharmaceutical composition of the present disclosure may be prepared into a unit dosage form or multiple dosage form along with a pharmaceutically acceptable carrier and/or excipient according to a method that can be easily employed by those skilled in the art. The formulation may be in the form of solution in oily or aqueous medium, suspension, emulsion, extract, dust, powder, granule, tablet or capsule, and may further include a dispersant or stabilizer.

In an exemplary embodiment of the present disclosure, the pharmaceutical composition of the present disclosure is formulated for application to skin. The formulation form is not particularly limited and may be, for example, powder, gel, ointment, cream, lotion, liquid or aerosol.

### EXAMPLES

The examples and experiments will now be described. The following examples and experiments are for illustrative purposes only and not intended to limit the scope of this disclosure.

Methods and Materials

1. Reagents and Instruments

Hydroquinone (HQ), epigallocatechin gallate (EGCG), 3,4-dihydroxy-L-phenylalanine (L-DOPA), dimethyl sulfoxide (DMSO), 2,6-di-tert-butyl-4-methylphenol (BHT), 1,1-diphenyl-2-picrylhydrazyl (DPPH), arbutin, mushroom tyrosinase, tannic acid, L-tyrosine, ascorbic acid, Folin-Ciocalteu phenol reagent and diethylene glycol were purchased from Sigma Chemical Company (USA). TRP1 was purchased from Santa Cruz (USA), and rutin was purchased from Acros (USA). Propylene glycol was purchased from OCI (Korea), and ketamine hydrochloride was purchased from Yu-Han Company (Korea). Hydrogen peroxide was purchased from Junsei (Japan). Hematoxylin, bovine serum albumin (BSA), retinoic acid and propylene glycol were purchased from Sigma Chemical Co. (St. Louis, USA). A UVB sunlamp (UVM-225D, Mineralight Lamp UVP, USA) was used for UVB radiation, a UV radiometer (HD 9021, Delta OHM, Italy) was used for UV measurement, and a mexameter (MX18, CK Electronic GmbH, Germany) was used for melanin index measurement. An inverted microscope (CKX41, Olympus, Japan) was used to observe cells, a, fluorescence microscope (Axio imager, Carl Zeiss, Germany) was used for morphology observation, and i-solution (IMT i-solution ver. 8.0, Canada) was used for image analysis. A corneometer (CM825, CK Electronic GmbH, Germany) and a tewameter (TM300, CK Electronic GmbH, Germany) were used for measurement of erythema index, water content and transepidermal water loss (TEWL). Visioline (VL650, CK Electronic GmbH, Germany) was used for wrinkle measurement, and an optical microscope (BX51, Olympus, Japan) and a digital camera system (C14 plus, Olympus, Japan) were used for histological observation. An auto hematology analyzer (Sysmex, XE-2100, Co Ltd., Japan) was used for histological examination.

2. Materials for Extraction

Green tea (GT), white tea (WT) and black tea (BT) were purchased at a medicinal herb market in Daegu, Korea. 600 g of GT, WT or BT was boiled for 2 hours together with 6 L of distilled water in a heating extractor (COSMOS-660, Kyungseo Machine Co., Korea) and then concentrated. Subsequently, the aqueous extract was freeze0dried into powder. Thus prepared sample was dissolved in a vehicle [propylene glycol:ethanol:water (5:3:2)] with various concentrations.

3. Measurement of Antioxidant Activity

3-1. Total Polyphenol Contents

Total polyphenol contents of AT, KA, EGCG, GT, WT and BT were measured according to the Folin-Denis assay (Folin and Denis, 1912). After adding 1 mL of the test sample dissolved in DMSO to a test tube and adding 1 mL of the Folin's reagent, the tube was allowed to stand for 3 minutes. Then, after adding 1 mL of 10% Na₂CO₃, the resulting mixture was shaken vigorously. After allowing the tube to stand for 60 minutes, absorbance was measured at 760 nm. The standard curve was prepared using tannic acid.

3-2. Total Flavonoid Contents

Total flavonoid contents of AT, KA, EGCG, GT, WT and BT were measured according to the modified method of Davis et al. (1980). After adding 1 mL of the test sample in a test tube, 10 mL of diethylene glycol and 1 mL of 1 N NaOH were added. The resulting mixture was shaken vigorously and absorbance was measured at 420 nm after reaction in warm water of 37° C. for 60 minutes. The standard curve was prepared using rutin.

3-3. Electron Donating Ability

DPPH radical scavenging effect was measuring according to the Blois' method (1958). AT, KA, HQ, EGCG, GT, WT or BT was dissolved in DMSO to concentrations of 100, 200, 400, 800, 1,600 and 3,200 μg/mL. After adding 1 mL of the test sample in a test tube, 4×10⁻⁴ M DPPH (4 mL) was added. The resulting mixture was shaken vigorously, allowed to stand in warm water of 60° C. for 10 seconds, and absorbance was measured at 525 nm. BHT was used as the positive control group. Then, free radical scavenging activity of each solution was calculated as “% inhibition” according to the following equation: % inhibition=[1−(Aₛₐₘₚₗₑ/A_{blank})]×100, wherein A_{b1ank} is the absorbance of the control group (excluding the test sample) and Aₛₐₘₚₗₑ is the absorbance of the test compound.

4. Cell Experiment

4-1. Cell Culture

Melan-a cells were purchased from Dr. Dorothy Bennett (St. George's Hospital, UK). Immortalized Melan-a cells with high pigment content are derived from C57BL/6 mouse. The cells were cultured using Roswell Park Memorial Institute (RPMI)-1640 medium containing 10% fetal bovine serum (FBS), 1% penicillin/streptomycin (P/S) and 200 nM 12-O-tetradecanoylphorbol 13-acetate (TPA) in a 10% CO₂ incubator at 37° C. for 72 hours.

4-2. MTT Assay

Melan-a cells were seeded on a 96-well plate (0.5×10⁴ cells/well) and cultured in a 10% CO₂ incubator at 37° C. for 24 hours. After adding 200 μL of AT, KA, EGCG, GT, WT or BT diluted to various concentrations (3.125, 6.25, 12.5, 25 and 50 μg/mL) using RPMI-1640 medium to the wells, the cells were cultured in a 10% CO₂ incubator at 37° C. for 48 hours. Then, the cells were cultured in a medium containing 0.5 μg/mL MTT in a 10% CO₂ incubator at 37° C. for 3 hours. Subsequently, after settling the cells by centrifuging the plate at 1,000 rpm for 10 minutes, the medium was removed. Then, 200 μL of DMSO was added and the cells were suspended for 15 minutes in a plate shaker. Absorbance was measured at 540 nm using an ELISA reader. Cell viability (%) was calculated according to the following equation: % cell viability=(Aₛₐₘₚₗₑ/A_{blank})×100, wherein A_{blank} is the absorbance of the control group (excluding the test sample) and Aₛₐₘₚₗₑ is the absorbance of the test compound.

4-3. Morphological Observation of Melan-a Cells

Melan-a cells were treated with AT, KA, EGCG, GT, WT or BT at 1.563, 3.125, 6.25 or 12.5 μg/mL and cultured in a 10% CO₂ incubator at 37° C. for 72 hours. Then, after exchanging the medium, the cells were observed under an inverted microscope at a magnification of ×200.

4-4. Melanin Analysis

Melan-a cells were seeded on a 96-well plate (2×10⁴ cells/well) and cultured in a 10% CO₂ incubator at 37° C. for 24 hours. Then, after adding 200 μL of AT, KA, EGCG, GT, WT or BT diluted to various concentrations (1.563, 3.125, 6.25 and 12.5 μg/mL) using RPMI-1640 medium to the wells, the cells were cultured in a 10% CO₂ incubator at 37° C. for 72 hours, and then washed. This procedure was repeated several times. After dissolving Melanin with 1N NaOH, absorbance was measured at 490 nm using an ELISA reader. Melanin content (%) was calculated according to the following equation: % melanin content=(Aₛₐₘₚₗₑ/A_{blank})×100, wherein A_{blank} is the absorbance of the control group (excluding the test sample) and Aₛₐₘₚₗₑ is the absorbance of the test compound.

4-5. Analysis of Intracellular Tyrosinase Activity

Melan-a cells were seeded on a 60 φ cell culture dish (4×10⁵ cells/well) and cultured in a 10% CO₂ incubator at 37° C. for 24 hours. After washing with PBS, the cells were suspended by adding 200 μL of 1% triton X-100 to the culture dish, transferred to an e-tube, and fixed on ice. 60 minutes later, after shaking for 2-3 seconds, the cells were centrifuged at 4° C. and 14,000 rpm for 20 minutes to obtain 50 μL of protein, which was mixed with 49 μL of 0.1 M phosphate buffer (pH 6.8) and 1 μL of AT, EGCG, GT, WT or BT (1.563, 3.125, 6.25 or 12.5 μg/mL). After allowing the mixture to stand for 1 hour and adding 100 μL of L-DOPA, the mixture was incubated in a 10% CO₂ incubator at 37° C. for 1 hour. Absorbance was measured at 490 nm using an ELISA reader. Tyrosinase activity was calculated according to the following equation: % inhibition=[1−(Aₛₐₘₚₗₑ/A_{blank})]×100, wherein A_{blank} is the absorbance of the control group (excluding the test sample) and Aₛₐₘₚᵢₑ is the absorbance of the test compound.

4-6. Analysis of Cell-Extracted Tyrosinase Activity

Melan-a cells were seeded on a 60 φ cell culture dish (4×10⁵ cells/well) and cultured in a 10% CO₂ incubator at 37° C. for 72 hours. After washing with PBS, the cells were suspended by adding 200 μL of 1% triton X-100 to the culture dish, transferred to an e-tube, and fixed on ice. 60 minutes later, after shaking for 2-3 seconds, the cells were centrifuged at 4° C. and 14,000 rpm for 20 minutes to obtain 50 μL of protein, which was mixed with 49 μL of 0.1 M phosphate buffer (pH 6.8) and 1 μL of AT, EGCG, GT, WT or BT (1.563, 3.125, 6.25 or 12.5 μg/mL). After allowing the mixture to stand for 1 hour and adding 100 μL of L-DOPA, the mixture was incubated in a 10% CO₂ incubator at 37° C. for 1 hour. Absorbance was measured at 490 nm using an ELISA reader. Tyrosinase activity was calculated according to the following equation: % inhibition=[1−(Aₛₐₘₚₗₑ/A_{blank})]×100, wherein A_{blank} is the absorbance of the control group (excluding the test sample) and Aₛₐₘₚₗₑ is the absorbance of the test compound.

4-7. Western Blotting Analysis

Cell lysate treated with 6.25 and 12.5 μg/mL CSWE was prepared by sonicating Melan-a cells in 0.1 M Tris-HCl (pH 7.2) buffer containing 1% Nonidet P-40, 0.01% SDS and protease inhibitor cocktail (Roche, Mannheim, Germany). Protein concentration in the cell lysate was measured using the Pierce protein assay kit (Pierce Biotechnology, Inc., Rockford, USA), using BSA as standard. After adding same quantity of protein (10 mg) on each lane, the proteins were separated by electrophoresis on 10% polyacrylamide gel. After transblotting onto nitrocellulose membrane, the membrane was incubated with PEP7 (1:10000 dilution, anti-tyrosinase antibody), PEP1 (1:10000 dilution, anti-TRP1 antibody) and PEP8 (1:10000 dilution, anti-TRP2 antibody). Then, the membrane was incubated with horseradish peroxidase-conjugated anti-rabbit IgG (1:1000 dilution; Amersham, Bucks, UK). Immunoreactive bands were detected by chemiluminescence using the ECL western blotting detector (Amersham, Bucks, UK). Band intensity was measured using the ImageJ program (NIH, Bethesda, USA). β-actin was used as internal control group for the immunoblotting.

4-8. RT-PCR Analysis

Total RNA was prepared using Trizol reagent (Invitrogen, Caylsbad, Calif.) according to the manufacturer's instructions. 5 μg of total RNA was subjected to reverse transcription with 8 μL of M-MLV RT 5× buffer, 3 μL of 10 mM dNTPs, 0.45 μL of 10,000 U RNase inhibitor, 0.3 μL of 50,000 U M-MLV reverse transcriptase (Promega, Madison, USA) and 1.5 μL of 50 μmol/μL oligo dT (Bioneer, Daejeon, Korea). Single-stranded cDNA was amplified by PCR in a reaction solution containing 4 μL of 5× green Go Taq flexi buffer, 0.4 μL of 10 mM dNTPs, 0.1 μL of 500 U Taq polymerase, 1.2 μL of 25 mM MgCl₂ (Promega, Madison, USA) and 0.4 μL of 20 μmol/μL sense and antisense primers of tyrosinase, TRP-1, TRP-2 or β-actin.

Sequences of the primers used in the PCR are as follows:

Tyrosinase:
forward primer (5′-CAT TTT TGA TTT GAG TGT CT-3′),
reverse primer (5′-TGT GGT AGT CGT CTT TGT CC-3′);
TRP-1:
forward primer (5′-GCT GCA GGA GCC TTC TTT CTC-3′),
reverse primer (5′-AAG ACG CTG CAC TGC TGG TCT-3′);
TRR-2:
forward primer (5′-GGA TGA CCG TGA GCA ATG GCC-3′),
reverse primer (5′-CGG TTG TGA CCA ATG GGT GCC-3′);
β-actin:
forward primer (5′-ACC GTG AAA AGA TGA CCC AG-3′),
reverse primer (5′-TAC GGA TGT CAA CGT CAC AC-3′).

Expected size of each PCR product of tyrosinase, TRP-1, TRP-2 and β-actin is 1192, 268, 1044 and 528 bp. PCR condition was as follows: tyrosinase and TRP-1: 28 cycles of denaturation at 94° C. for 60 seconds, annealing at 56° C. for 60 seconds and extension at 72° C. for 60 seconds; β-actin: 30 cycles of denaturation at 94° C. for 30 seconds, annealing at 51° C. for 30 seconds and extension at 72° C. for 60 seconds. The PCR product was analyzed on 1.2% agarose gel. β-actin was used as internal control group for comparison of relative expression level with tyrosinase, TRP-1 and TRP-2.

5. Animal Experiment (Whitening Effect)

5-1. Test Animal

Three brown guinea pigs weighing 450-550 g were purchased from Oriental Yeast Co. Ltd. (OYC; Japan). The animals were accustomed to the test environment for 7 days. The animals were housed in individual cages under the environment of 22±1° C. and 50±5% relative humidity with 12-hour alternate dark/light cycles. Feed and water were given ad libitum. After administering the test sample for 4 weeks, the animal was anesthetized with ketamine hydrochloride, and the melanin pigmented area was sampled using a biopsy punch (φ12 mm). The sample was fixed in neutral-buffered 10% formalin and embedded in paraffin for histological observation.

Test groups were as follows:

Normal group (N): saline administration group

Control group (Control, C): UVB radiation+saline administration group

Vehicle control group (VC): UVB radiation+vehicle administration group

Positive control group (HQ): UVB radiation+2% HQ administration group

Test group 1 (EGCG): UVB radiation+2% EGCG administration group

Test group 2 (GT): UVB radiation+2% GT administration group

Test group 3 (WT): UVB radiation+2% WT administration group

Test group 4 (BT): UVB radiation+2% BT administration group

5-2. UVB Radiation

UVB-induced hyperpigmentation was elicited on the back of the brown guinea pig according to the method of Choi et al. (2004). After anesthetizing the guinea pig with ketamine hydrochloride (100 mg/kg BW), 24 separate areas (φ 12 mm) on the skin of the back was exposed to UVB radiation (302-nm sunlamp). The areas were exposed to UVB of 500 mJ/cm² for 3 consecutive weeks, once a week. That is, a total of 1,500 mJ/cm² of UVB was radiated.

5-3. Application of Test Compound

On day 10 after the final UVB radiation, 30 μL (1.06 mg/cm²/day) of 2% HQ, EGCG, GT, WT or BT was topically applied for 4 weeks to the hyperpigmented areas (9 areas per each) using a micropipette, twice a day, and 4 days a week. Saline was used as control, and propylene glycol:ethanol:water (5:3:2) was used as vehicle control.

5-4. Gross Observation of Depigmentation

The melanin hyperpigmented areas were observed by gross observation once a week. The whitening effect of each test group was compared with the control group. The comparison was repeated every week for 4 weeks, and the skin surface was photographed every week.

5-5. Measurement of Melanin Index

Before and after the topical application of the test sample, change in melanin pigmentation was measured 3 times in a non-invasive manner using a mexameter, once a week. The degree of pigmentation was analyzed by averaging the measured values.

5-6. Morphological Observation of Skin Tissue

In order to observe the morphological change of the skin tissue using an optical microscope, the extracted skin tissue was fixed in a 10% neutral formalin solution for 12 hours and washed with flowing water. After dehydrating in 70, 80, 95 and 100% ethanol followed by washing with xylene, the tissue was embedded in paraffin and 4 μm-thick samples were prepared using a microtome.

5-6-1. Hematoxylin and Eosin Staining

Paraffin was removed from the sample using xylene. The sample was washed with flowing water. After staining the nucleus for 5 minutes in Harris hematoxylin solution, the sample was washed with flowing water, precipitated 3 times in 1% HCl alcohol solution, and stained blue with 1% ammonia solution. After staining the cytoplasm in eosin solution for 3 minutes, the sample was transferred to 80, 95, 95, 100 and 100% alcohol for dehydration. After washing, the sample was put in Canada balsam for microscopic observation.

5-6-2. Fontana-Masson's Silver Staining

Paraffin was removed from the sample using xylene. After staining the sample for 60 minutes in silver nitrate solution at 56° C. according to the Masson's method (1928), followed by washing, color-toning 0.2% gold chloride solution and washing again, the sample was kept in 5% sodium thiosulfate solution for 5 minutes. Subsequently, after washing the sample once again, followed by counterstaining in nuclear fast red solution, dehydration in 95%, 95%, 100% and 100% alcohol and washing with xylene, the sample was put in Canada balsam for microscopic observation. The percentage of the melanin-pigmented area was calculated using an image analysis software.

5-6-3. Immunohistochemical Staining

In order to observe the degree of melanocyte and melanosome production, the extracted skin tissue was fixed in 10% neutral formalin solution for 12 hours and washed with flowing tap water. After dehydration, followed by washing with xylene and embedding in paraffin, 4 μm-thick samples were prepared. The sample was adhered on a coating slide and stained using the BenchMark XT automated immunostainer (Ventana Medical Systems, USA) after removing paraffin. For automated process, the sample was washed using reaction buffer and kept in 3% H₂O₂ solution for 3 minutes for inhibition of peroxidase activity. Following reaction with S-100 and HMB-45 primary antibodies diluted to 1:100, the sample was washed with reaction buffer, reacted with biotin for 20 minutes, and then reacted with streptavidin for 25 minutes. After coloring with diaminobenzidine (DAB) using the Ventana detection kit), the sample was stained for observation using an optical microscope. In order to compare the degree of melanocyte and melanosome production, the percentage of the area of S-100 and gp 100 proteins was calculated using an image analysis software.

6. Animal Experiment (Wrinkle Improving Effect)

6-1. Test Animal

7-week-old SKH-1 hairless mice was purchased from Charles River (Japan). The animals were accustomed to the test environment for 1 week. The animals were housed in individual cages under the environment of 22±1° C. and 50±5% relative humidity with 12-hour alternate dark/light cycles. Feed and water were given ad libitum. The animals were grouped into 7 groups as follows, with 7 mice per each:

Normal group (N): no application group;

Control group (C): saline administration group;

Positive control group (RA): 0.01% retinoic acid administration group;

Test group 1 (GT): 2% green tea administration group;

Test group 2 (WT): 2% white tea administration group;

Test group 3 (BT): 2% black tea administration group;

On week 4, after fasting the mouse for 4 hours, blood sample was collected from the inferior vena cava under ether anesthesia. After extracting the skin, a portion of the skin was fixed in 10% neutral-buffered formalin solution for histological analysis and the remainder was kept in a refrigerator (−80° C.) for analysis of MMPs. Thymus and spleen extracts were washed with saline and weighed on an electronic scale after removing water with filer paper.

6-2. UVB Radiation

The mouse was subjected to UVB radiation (302 nm sunlamp) on the back for 4 weeks, 3 times a week. The radiation amount was 1 minimal erythemal dose (MED: 60 mJ/cm²) on the 1st week, 2 MED (120 mJ/cm²) on the 2nd week, 3 MED (180 mJ/cm²) on the 3rd week, and 4 MED (240 mJ/cm²) on the 4th to 12th weeks. The radiation with 4 MED was carried out once a week during the test period.

6-3. Application of Test Sample

After inducing wrinkle formation, 200 μL of saline or the sample was applied for 4 weeks, 5 times a week (2%: 0.26 g/kg BW/day). RA was applied together with polyethylene glycol at a concentration of 0.01% for 4 weeks, 5 times a week, twice a day, with 200 μL per each.

6-4. Measurement of Skin Erythema Index, Water Content and Transepidermal Water Loss

Skin erythema index, water content and transepidermal water loss (TEWL) were measured in a non-invasive manner using a mexameter, a corneometer and a tewameter, once a week during the test period.

6-5. Measurement of Change in Water and Feed Intake, Feed Efficiency and Body Weight

Water and feed intake of the test animal was measured once a week. Body weight was measured between 09:00 and 10:00 in the morning, once a week during the test period.

7. Morphological Observation of Skin Tissue (Wrinkle Improving Effect)

7-1. Measurement of Wrinkles

Skin replica was prepared from the back skin of the hairless mouse using the Silflo impression material (Flexico, England). The roughness of the skin was assessed using Visioline. Total wrinkle area (mm²) was calculated.

7-2. Histological Analysis

7-2-1. Hematoxylin & Eosin Staining

After fixing in 10% neutral-buffered formalin solution at room temperature for 24 hours, the excised skin tissue was washed, dehydrated, cleaned, embedded in paraffin and sliced into 4 μm-thick sections. The section was stained with H&E and pattern change of the skin tissue was observed using an optical microscope.

7-2-2. Masson's Trichrome Staining

After fixing in 10% neutral-buffered formalin solution at room temperature for 24 hours, the excised skin tissue was washed, dehydrated, cleaned, embedded in paraffin and sliced into 4 μm-thick sections. The section was immersed in Bouin solution at room temperature overnight and stained with Masson's trichrome. Quantity and shape of collagen fibers were observed under an optical microscope.

7-2-3. Verhoeff Staining

After fixing in 10% neutral-buffered formalin solution at room temperature for 24 hours, the excised skin tissue was washed, dehydrated, cleaned, embedded in paraffin and sliced into 4 μm-thick sections. The section was stained and washed with Verhoeff's solution and identified and washed with 2% ferric chloride. Then, the section was treated with sodium trisulfate. Loss of elastin fibers and degenerative change in the skin were observed under an optical microscope.

7-2-4. Toluidine Blue Staining

After fixing in 10% neutral-buffered formalin solution at room temperature for 24 hours, the excised skin tissue was washed, dehydrated, cleaned, embedded in paraffin and sliced into 4 μm-thick sections. The section was stained and washed with toluidine blue solution, and mast cell distribution pattern and degree of degranulation of the skin and the hypodermis were observed under an optical microscope.

8. Measurement of Gene Expression in Skin Tissue

8-1. Reverse Transcription Polymerase Chain Reaction

50 mg of the sample from the mouse back skin kept at low temperature was homogenized with 1 mL of Trizol. The homogenized sample was incubated at room temperature for 5 minutes after adding 200 μL of chloroform, and centrifuged at 15,000 rpm and 4° C. for 10 minutes. After collecting and transferring the supernatant to a fresh tube and adding 500 μL of isopropyl alcohol, the sample was centrifuged at 4° C. and 15,000 rpm for 15 minutes. The resulting RNA pellet was washed with 1 mL of 70% ethanol and centrifuged at 4° C. and 15,000 rpm for 15 minutes. The RNA pellet finally obtained by removing the supernatant was diluted with diethylpyrocarbonate (DEPC) and quantitated with 1.8<optical density (OD; A₂₆₀/A₂₈₀)<2.0 using a white light/UV transilluminator. cDNA was synthesized from total RNA at a concentration of 0.1-1 μg/μL using the Cycle Script RT PreMix (dT20) kit. The cDNA synthesis was performed by 12 cycles of 1 minute at 30° C. and 4 minutes at 50° C. in 205 μL of DEPC, and the sample was heated at 95° C. for 5 minutes. Polymerase chain reaction (PCR) was carried out in 15.2 μL of sterilized water containing 2 μL of template and 10 μmol/μL of primer using the Accupower™ PCR PreMix kit. The reaction was carried out for 35 cycles. The primer used to amplify each fragment is shown in Table 1. The PCR product was separated by electrophoresis on 1.5% agarose gel and visualized using ethidium bromide. DNA band density was calculated using the Kodak Gel Logic 100 image analysis system.

TABLE 1

| | | Expected |
|---|---|---|
| Items | Primers | size(bp)³⁾ |
| MMP-3¹⁾ | Forward | 317 |
| | (5′→3′) TAGCAGGTTATCCTAAAAGCA | |
| | Reverse | |
| | (5′→3′) CCAGCTATTGCTCTTCAAT | |
| GAPDH²⁾ | Forward | 478 |
| | (5′→3′) CCCACTAACATCAAATGGGG | |
| | Reverse | |
| | (5′→3′) ACACATTGGGGGTAGGAACA | |

¹⁾MMP-3: Matrix metalloproteinase-3
²⁾GAPDH: Glyceraldehyde-3-phosphate dehydrogenase
³⁾bp: basepair

9. Zymography for MPP-2 and MPP-9

50 mg of skin tissue was weighed and homogenized by adding 400 mL of 1× PiPA lysis buffer. After centrifuging at 14,000 rpm for 15 minutes, the supernatant was used for test. The protein was quantitated using the BCA protein analysis kit (Pierce, USA). According to the Invitrogen's protocol, the tissue sample was prepared with a total volume of 10 μL including 5 μL of tris-glycine SDS sample buffer and 0.5 μg/μL of protein. For electrophoresis, after loading the sample into 10% zymogram gel (Novex, USA), the remaining portion was filled with distilled water. After incubation in 1× renaturing buffer, followed by incubation in 1× spreading buffer for 30 minutes, the buffer solution was discarded and the sample was further incubated at 37° C. for 21 hours. Subsequently, the gel was stained with Simply Blue™ Safe-Stain.

10. Hematological Analysis

Blood sample was added to a blood collection tube containing the anticoagulant K₂EDTA and mixed for more than 5 minutes. Then, concentration of erythrocytes, leukocytes, neutrophils, eosinophils, basophils, lymphocytes, monocytes, platelets and hemoglobin and hematocrit were measured using a hematological analyzer.

11. Statistical Analysis

Difference in numerical values between the groups was statistically evaluated by one-way analysis of variance (ANOVA), and Duncan's multiple range test was carried out using SPSS (v 17.0) for post-hoc test. The standard for statistical significance was set as p<0.05, p<0.01 and p<0.001.

Experimental Result 1: Whitening Effect

1. Antioxidant Activity

1-1. Total Polyphenol Contents

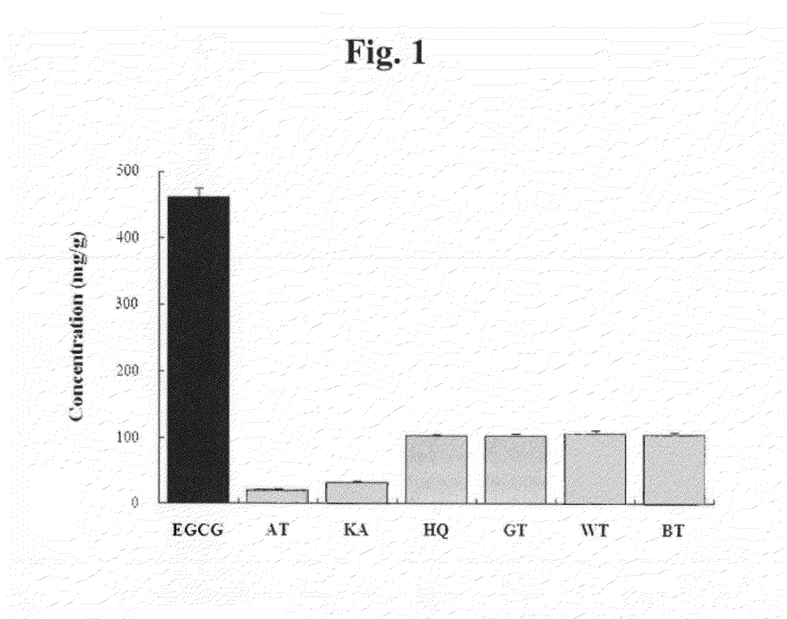

Total polyphenol contents of EGCG, AT, KA, HQ, GT, WT and BT measured using the standard curve for tannic acid were 462, 20, 31, 102, 104, 107 and 105 mg/g, respectively (see FIG. 1).

1-2. Total Flavonoid Contents

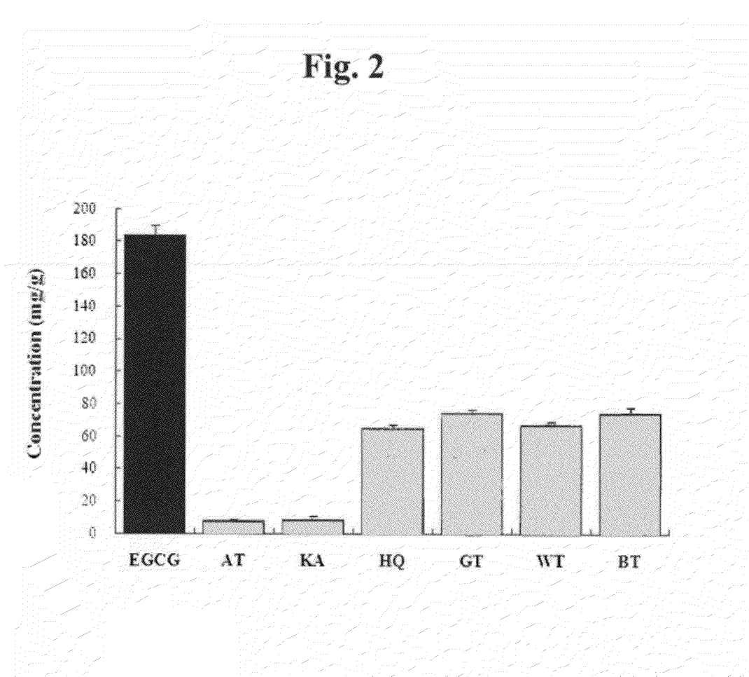

Total flavonoid contents of EGCG, AT, KA, HQ, GT, WT and BT measured using the standard curve for rutin were 184, 8, 9, 65, 75, 67 and 75 mg/g, respectively (see FIG. 2).

1-3. Electron Donating Ability

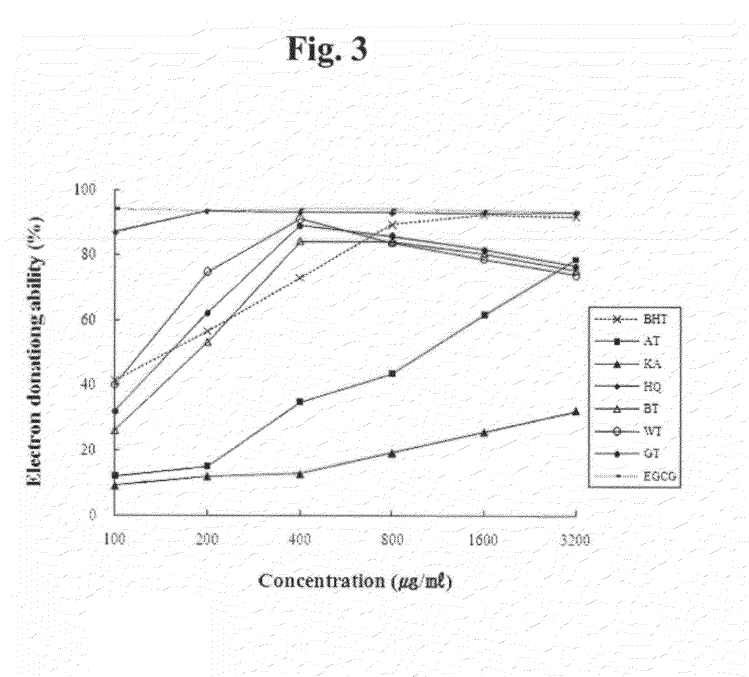

Electron donating ability of AT, KA, HQ, BT, WT, GT and EGCG at 800 μg/mL was 44, 19, 93, 84, 84, 86 and 94%, respectively. The ability of BHT was 89% at the same concentration. The electron donating ability of EGCG, GT, WT and BT was much higher than KA or AT and comparable to HQ (see FIG. 3).

2. Cell Experimental Result

2-1. Cell Viability

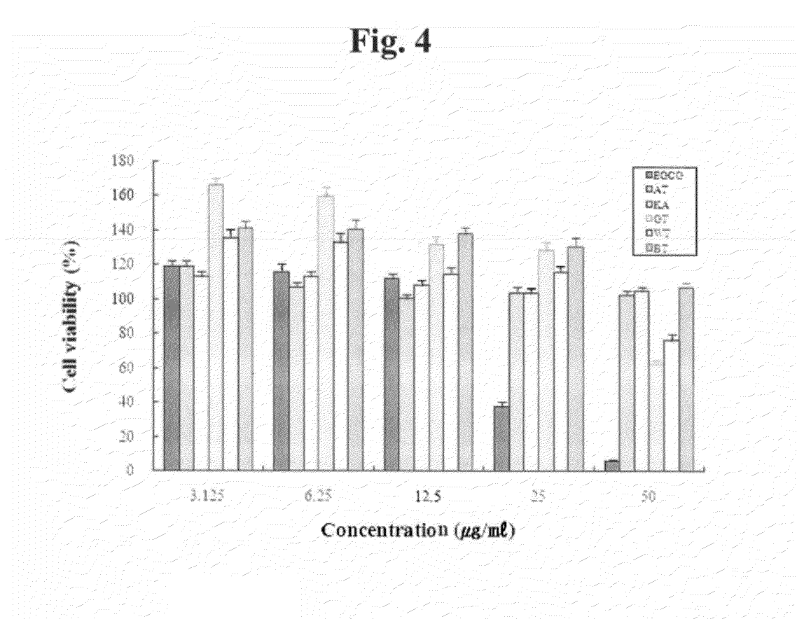

AT, KA and BT had no effect on viability of the Melan-a cells at 3.125 and 50 μg/mL, suggesting that the maximum permissible level (MPL) exceeds 50 μg/mL. On the other hand, GT and WT showed 38% and 24% decreased cell viability, respectively, with the MPL being 25 μg/mL. The MPL of EGCG was 12.5 μg/mL (see FIG. 4).

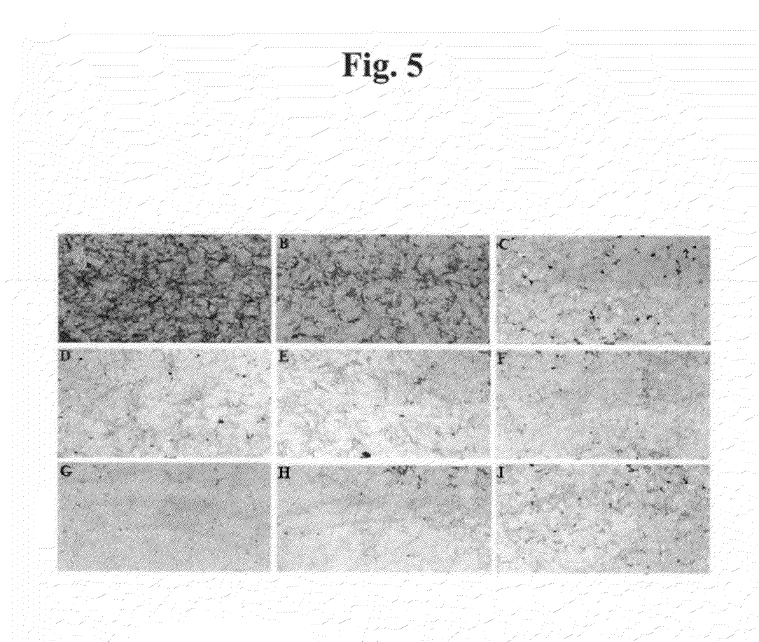

2-2. Morphological Observation of Melan-a Cells

Whereas a number of melanocyte dendrites and black precipitates induced by melanin synthesis were observed for the control group cells, the groups treated with GT, WT and BT showed dose-dependent decrease of the dendrites because of inhibited melanin synthesis (see FIG. 5). The inhibitory effect was in the order of BT >WT >GT.

2-3. Melanin Synthesis

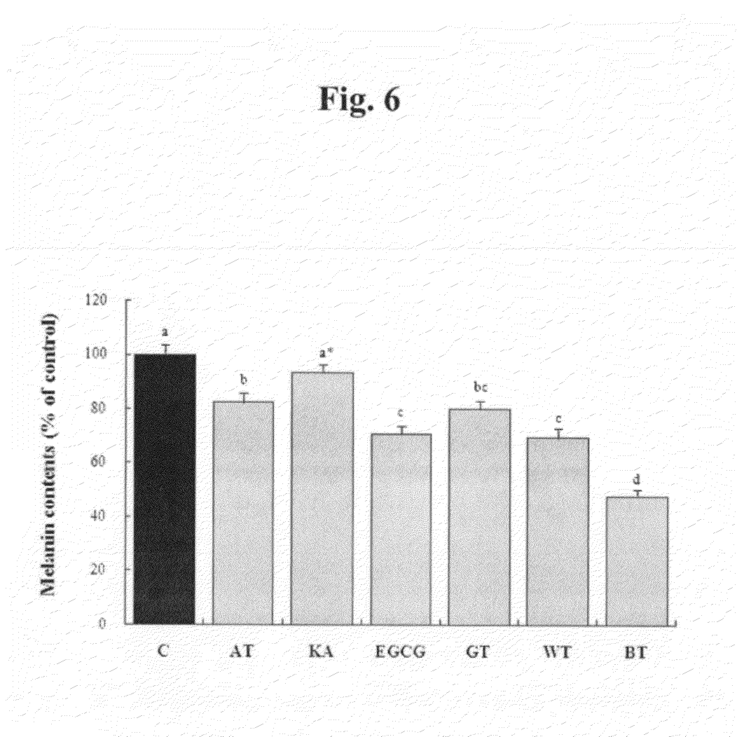

When compared with the control group, the groups treated with 12.5 μg/mL AT, KA, EGCG, GT, WT and BT showed melanin content decreased by 18% (p<0.001), 7% (p <0.05), 29% (p <0.001), 20% (p <0.001), 31% (p <0.001) and 52% (p <0.001), respectively (see FIG. 6).

2-4. Tyrosinase Activity

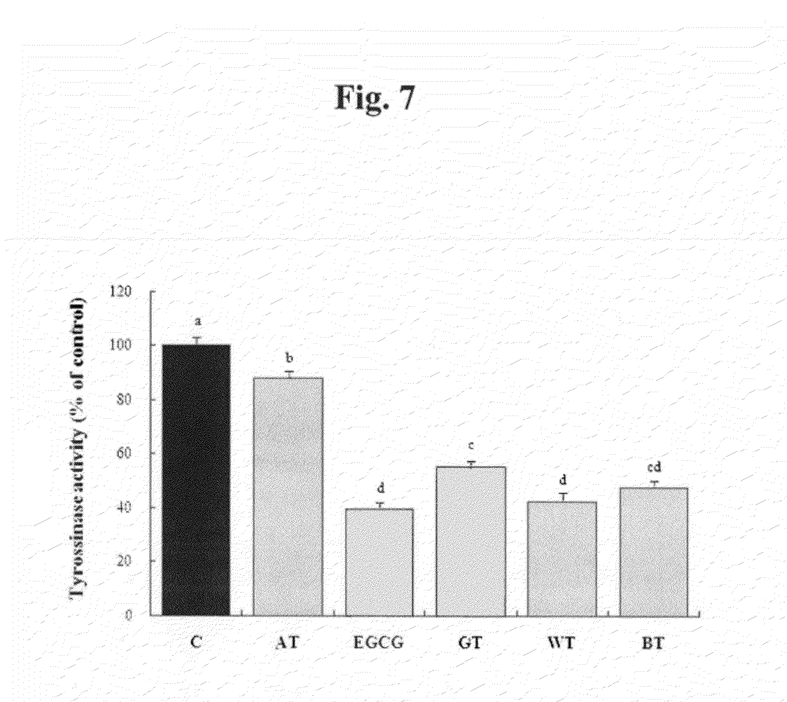

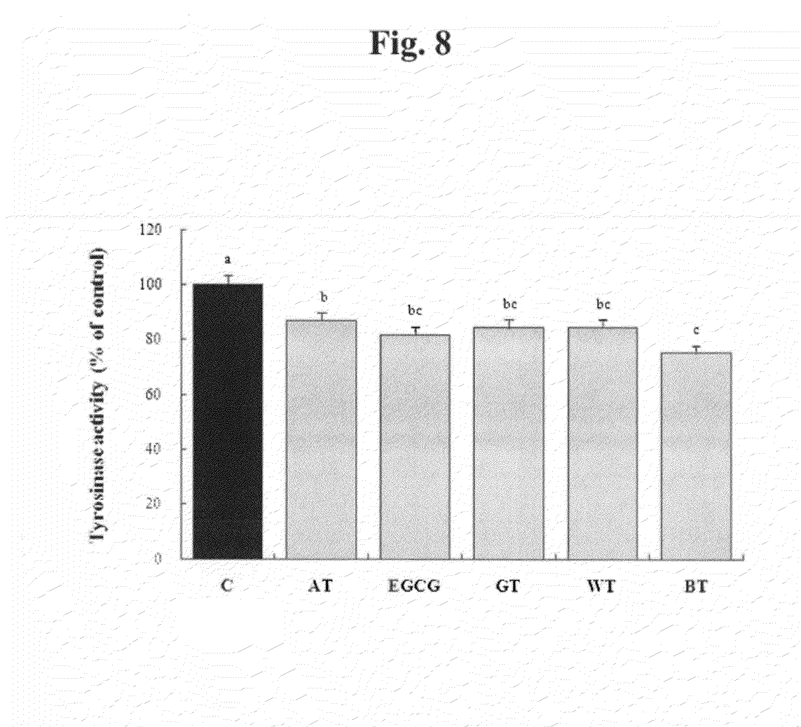

When compared with the control group, the groups treated with 12.5 μg/mL AT, EGCG, GT, WT and BT showed intracellular tyrosinase activity decreased by 12, 61, 45, 58 and 52% (p <0.001), respectively (see FIG. 7). And, when compared with the control group, the groups treated with 12.5 μg/mL AT, EGCG, GT, WT and BT showed tyrosinase activity in cell extract by 13, 18, 16, 15 and 25% (p <0.001), respectively (see FIG. 8).

2-5. Western Blotting Analysis

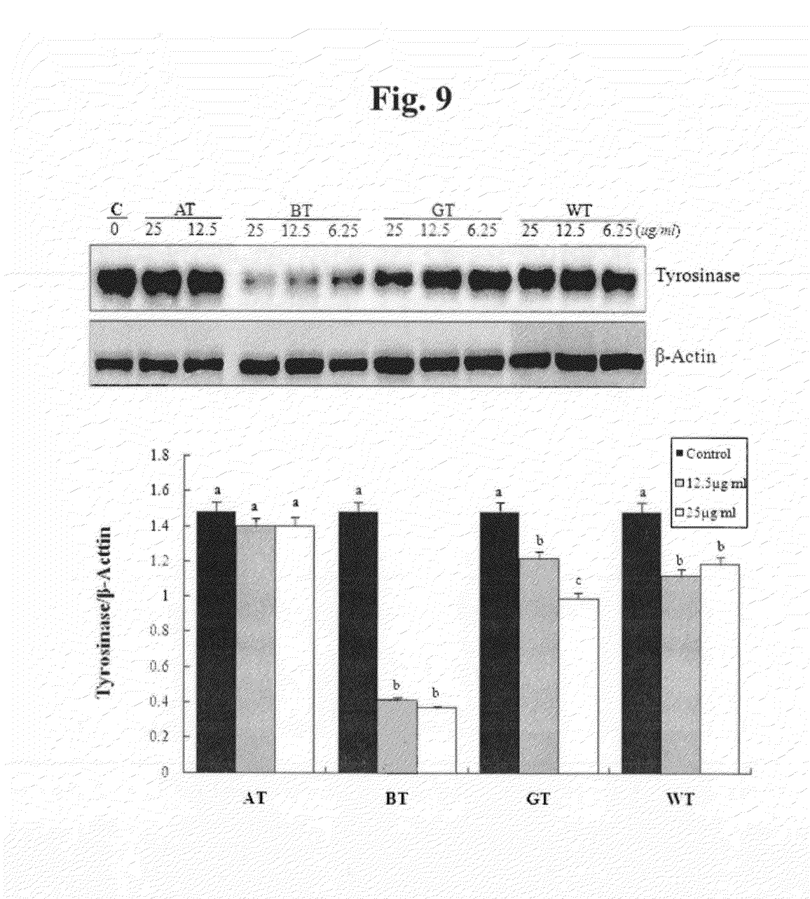

The test groups treated with black tea, green tea and white tea showed dose-dependent decrease in the expression of tyrosinase protein (see FIG. 9). When compared with the control group, the groups treated with 12.5 μg/mL black tea, green tea and white tea showed decreased expression of tyrosinase protein by 72, 18 and 25%, respectively (p <0.001). In contrast, the group treated with arbutin showed no significant decrease in the expression.

2-5. RT-PCR Analysis

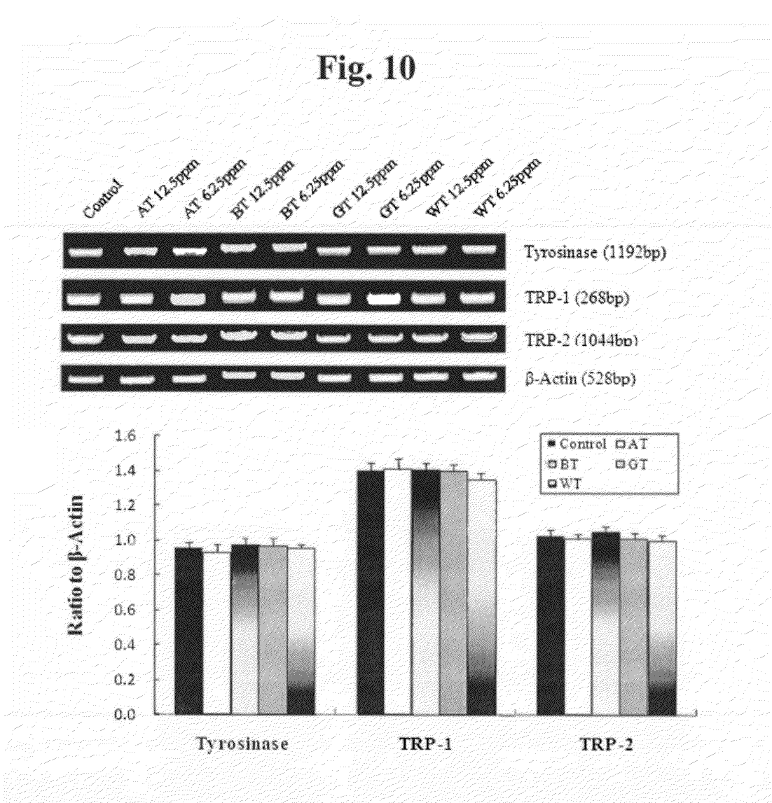

When compared with the control group, none of the test samples showed any appreciable effect on the expression of mRNA of tyrosinase, TRP-1 and TRP-2 (see FIG. 10).

3. Animal Experiment Result

3-1. Gross Observation of Depigmenting Effect

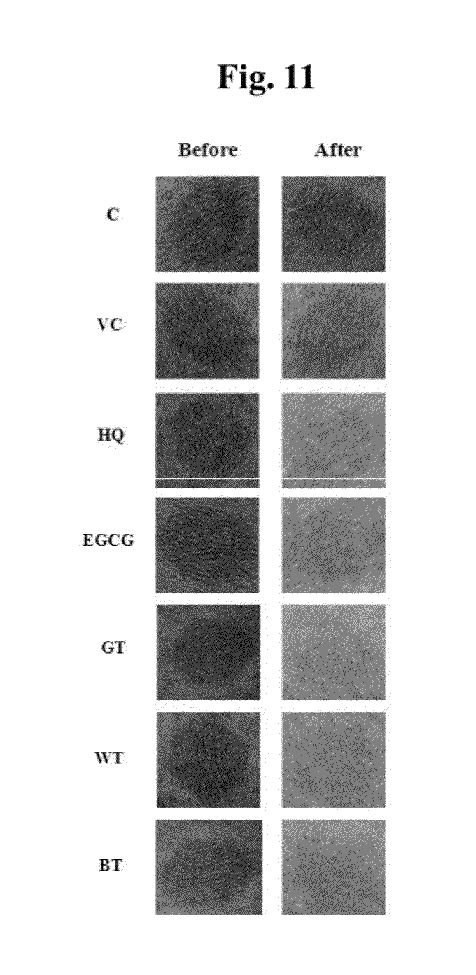

Melanin pigmentation was induced on the skin of guinea pig of all the test and control groups by radiating UVB continuously from 1st to 3rd weeks. During the test period, no negative side effect associated with the application of the test sample was observed. On week 2 after the topical application, depigmentation of the hyperpigmented started in the groups PC and E and the depigmentation was visible on week 4 (see FIG. 11).

3-2. Change in Melanin Index

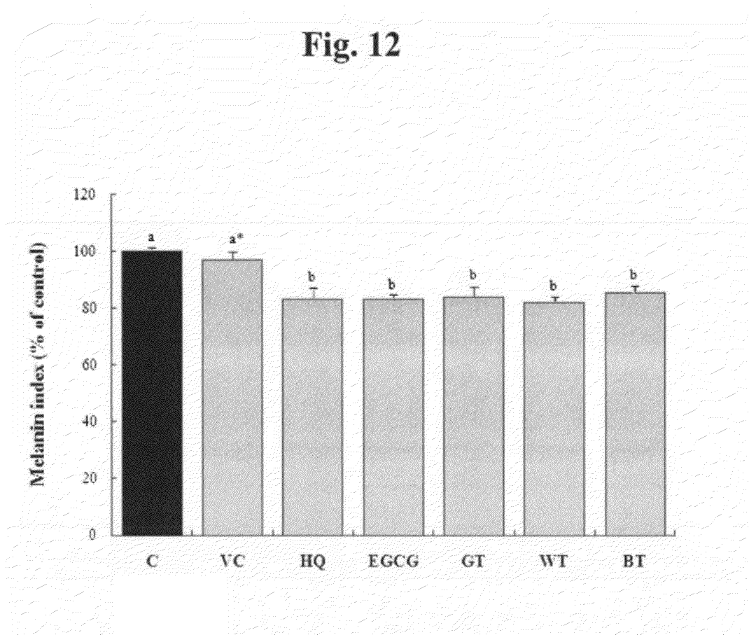

On week 1, the melanin index decreased remarkably in all groups except for the group VC (p <0.05). On week 2, the melanin index decreased greatly in PC and all E groups as compared to the group C (p <0.001). On week 4, the melanin index of the groups HQ, EGCG, GT, WT and BT decreased by 17, 17, 16, 18 and 15%, respectively, as compared to the group C (p <0.001) (see Table 2 and FIG. 12). The melanin index of the group VC decreased by 3% (p <0.05).

TABLE 2

| Wk | C | VC | HQ | EGCG | GT | WT | BT |
|---|---|---|---|---|---|---|---|
| 0 | 725.6 ± 14.4^{a} | 723.6 ± 18.5^{a} | 724.8 ± 22.8^{a} | 723.5 ± 11.3^{a} | 722.1 ± 21.0^{a} | 725.3 ± 12.8^{a} | 723.5 ± 13.4^{a} |
| 1 | 723.9 ± 14.0^{a} | 719.3 ± 18.2^{a} | 702.9 ± 24.4^{a}* | 704.1 ± 12.5^{a}* | 700.8 ± 20.3^{a}* | 700.2 ± 15.5^{a}* | 705.6 ± 12.0^{a}* |
| 2 | 721.6 ± 12.9^{a} | 709.8 ± 17.7^{ab} | 673.9 ± 26.1^{c} | 675.9 ± 12.4^{c} | 670.9 ± 21.5^{c} | 668.0 ± 15.1^{c} | 686.7 ± 16.2^{bc} |
| 3 | 712.2 ± 7.6^{a} | 697.8 ± 16.3^{a} | 636.7 ± 25.8^{b} | 636.6 ± 12.2^{b} | 653.0 ± 24.1^{b} | 627.4 ± 13.6^{b} | 650.9 ± 17.8^{b} |
| 4 | 701.2 ± 9.1^{a} | 679.9 ± 20.0^{a}* | 583.0 ± 28.0^{b} | 582.9 ± 12.8^{b} | 588.5 ± 24.2^{b} | 575.0 ± 14.9^{b} | 598.7 ± 18.3^{b} |

Values are mean ± SD of 9 samples.
Unit: AU (Arbitrary Unit)
Values with different superscript in the same row are significantly different (p < 0.001) by ANOVA and Duncan's multiple range tests.
*p < 0.05 compared to the C group by ANOVA and Duncan's multiple range tests.

3-3. Histological Change in Skin Tissue

3-3-1. Hematoxylin & Eosin Staining

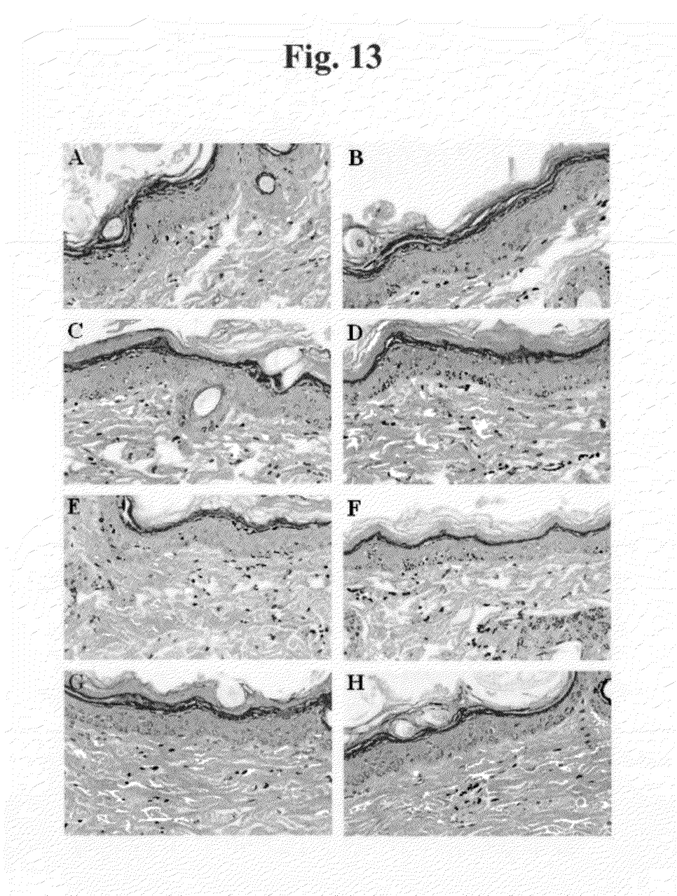

The epithelium of the control group was slightly thicker than those of other test groups. However, no inflammation or other undesirable effect associated with the application of the test sample was observed (see FIG. 13).

3-3-2. Fontana-Masson's Silver Staining

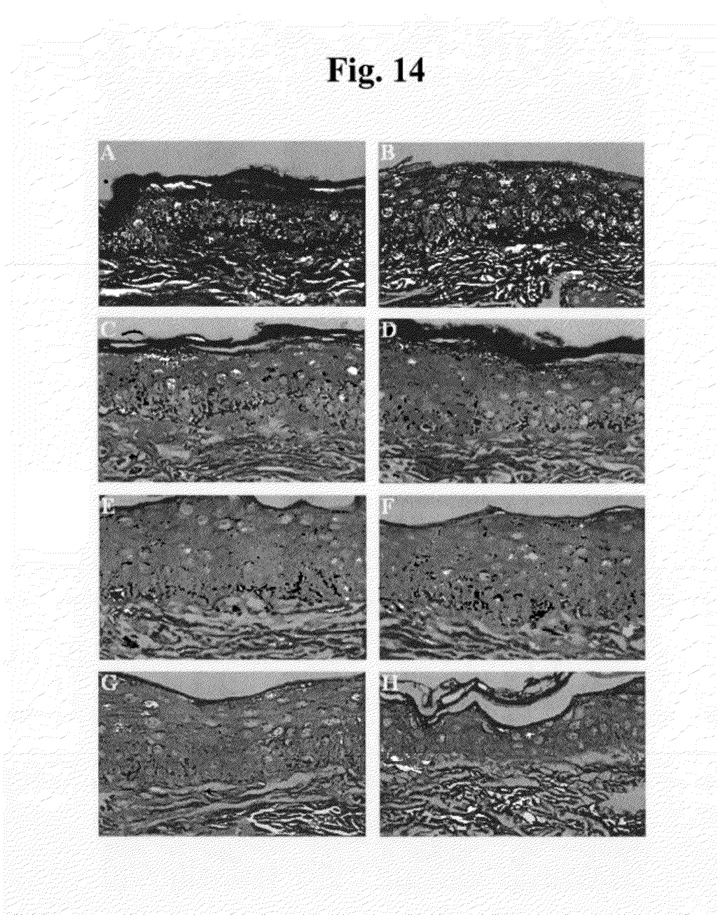

On week 4, the distribution of melanin granules was greatly increased in the groups C and VC as compared to the group N. The melanin granules were distributed from the basal layer to the stratum corneum. Meanwhile, the groups PC and E showed greatly decreased melanin granules in the epithelium as compared to the groups C and VC (see FIG. 14). An image analysis software was used to more objectively analyze the pigmented area in the epithelium.

The group C (20%) and the group VC (18%) showed greatly increased melanin pigmentation as compared to the group N (p <0.001). In contrast, the melanin pigmented area of the groups PC, EGCG, GT, WT and BT was greatly decreased by 53, 48, 51, 66 and 42%, respectively, as compared to the control group. The group VC showed a great decrease to 12% (p <0.05) (see Table 3).

TABLE 3

| Area | N | C | VC | HQ | EGCG | GT | WT | BT |
|---|---|---|---|---|---|---|---|---|
| Total | 10494 ± 2591 | 11705 ± 1461 | 11399 ± 1547 | 12116 ± 1709 | 11387 ± 1780 | 12079 ± 1687 | 12458 ± 1984 | 12224 ± 1809 |
| (μm²) | | | | | | | | |
| Melanin | 574 ± 154 | 2358 ± 439 | 2018 ± 447 | 1163 ± 268 | 1174 ± 189 | 1203 ± 328 | 852 ± 231 | 1441 ± 376 |
| pigment | | | | | | | | |
| (μm²) | | | | | | | | |
| % of | 5.6 ± 1.5^{a} | 20.1 ± 2.3^{d} | 17.7 ± 2.7^{d}* | 9.5 ± 1.5^{bc} | 10.5 ± 2.0^{c} | 9.9 ± 2.1^{bc} | 6.9 ± 1.8^{ab} | 11.7 ± 2.0^{c} |
| melanin | | | | | | | | |
| pigment | | | | | | | | |

Values are mean ± SD of 9 samples.
Values with different superscripts in the same row are significantly different (p < 0.001) by ANOVA and Duncan's multiple range tests.
*p < 0.05 compared to the C group by ANOVA and Duncan's multiple range tests.

3-3-3. Immunohistochemical Staining

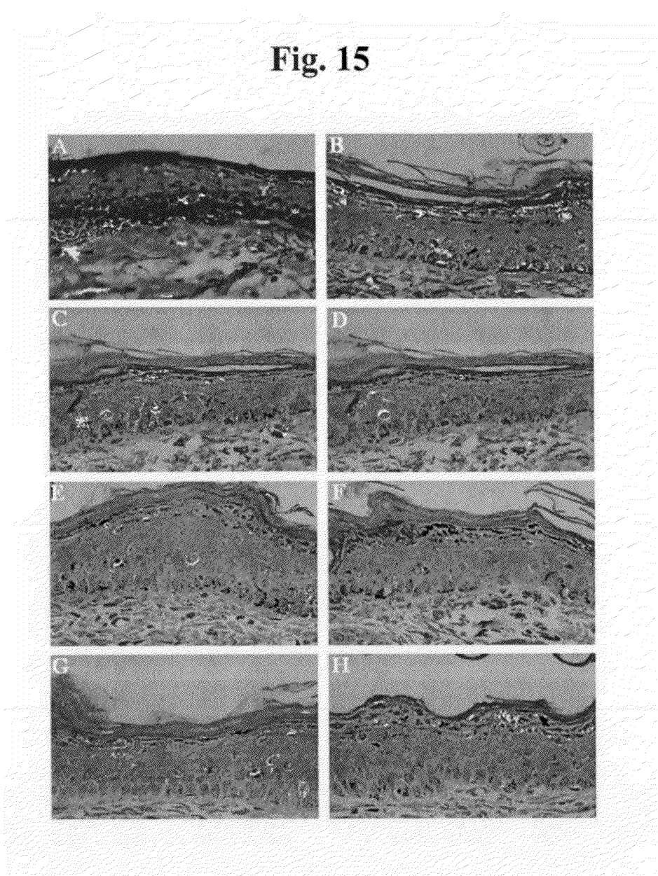

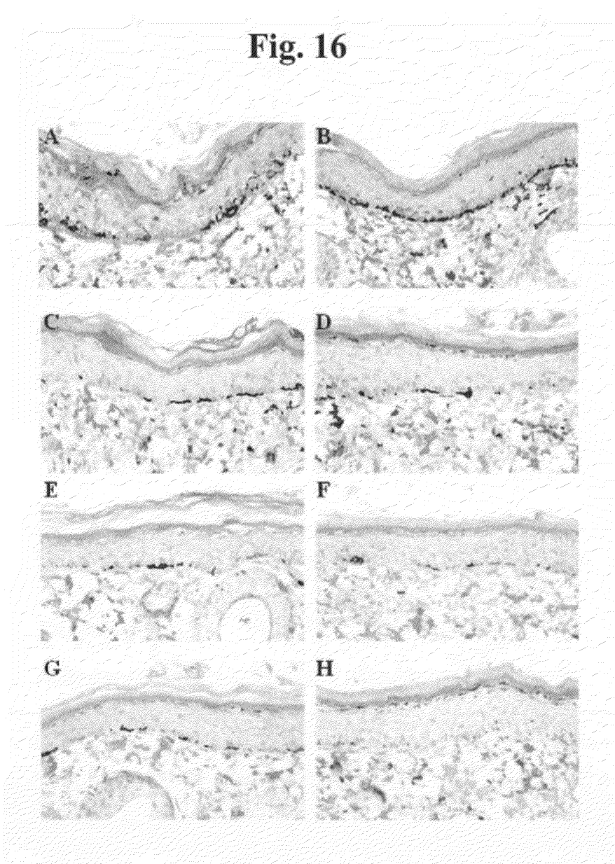

On week 4, the groups C and VC showed increased expression and densification of S-100 protein and gp100 protein as compared to the group N. In contrast, the groups PC and E showed distinct decrease as compared to the groups C and VC (see FIGS. 15-16). An image analysis software was used to more objectively analyze the area of S-100 and gp100 protein expression in the epithelium. The group C (14%) and the group VC (12%) showed greatly increased area of N S-100 protein expression as compared to the group (p <0.001), and the group VC showed 16% decrease as compared to the group C (p <0.001). The groups HQ, EGCG, GT, WT and BT showed decrease by 69, 62, 69, 73 and 58% as compared to the control groups (p <0.001) (see Table 4).

TABLE 4

| Area | N | C | VC | HQ | EGCG | GT | WT | BT |
|---|---|---|---|---|---|---|---|---|
| Total | 9078 ± 1760 | 11696 ± 2210 | 11768 ± 1039 | 11904 ± 1344 | 11676 ± 1790 | 12380 ± 1133 | 11411 ± 1803 | 11924 ± 1380 |
| (μm²) | | | | | | | | |
| S-100 | 259 ± 51 | 1648 ± 404 | 1379 ± 235 | 511 ± 89 | 611 ± 81 | 538 ± 67 | 431 ± 97 | 718 ± 208 |
| Protein | | | | | | | | |
| (μm²) | | | | | | | | |
| % of | 2.9 ± 0.5^{a} | 14.0 ± 1.3^{e} | 11.7 ± 1.3^{d} | 4.3 ± 0.5^{ab} | 5.3 ± 0.7^{bc} | 4.4 ± 0.7^{ab} | 3.8 ± 0.8^{ab} | 5.9 ± 1.1^{c} |
| S-100 | | | | | | | | |
| Protein | | | | | | | | |

Values are mean ± SD of 9 samples.
Values with different superscripts in the same row are significantly different (p < 0.001) by ANOVA and Duncan's multiple range tests.

The groups C and VC showed greatly increased area of gp 100 protein expression by 14% and 11%, respectively, as compared to the group N (3%) (p<0.001), and the group VC showed a great decrease of 18% as compared to the group C (p <0.001). The groups HQ, EGCG, GT, WT and BT showed decrease by 68, 61, 70, 71 and 59%, respectively, as compared to the group C (see Table 5).

TABLE 5

| Area | N | C | VC | HQ | EGCG | GT | WT | BT |
|---|---|---|---|---|---|---|---|---|
| Total | 10088 ± 1499 | 11592 ± 1697 | 10984 ± 1012 | 10579 ± 1308 | 11402 ± 1012 | 11802 ± 1286 | 11067 ± 1088 | 10367 ± 1202 |
| (μm²) | | | | | | | | |
| Melanin | 261 ± 67 | 1568 ± 234 | 1219 ± 186 | 469 ± 111 | 600 ± 107 | 478 ± 75 | 451 ± 97 | 580 ± 116 |
| pigment | | | | | | | | |
| (μm²) | | | | | | | | |
| % of | 2.6 ± 0.6^{a} | 13.6 ± 1.5^{e} | 11.1 ± 1.2^{d} | 4.4 ± 0.8^{c} | 5.3 ± 0.8^{c} | 4.1 ± 0.5^{ab} | 4.0 ± 0.6^{ab} | 5.6 ± 0.8^{c} |
| Melanin | | | | | | | | |
| pigment | | | | | | | | |

Values are mean ± SD of 9 samples.
Values with different superscripts in the same row are significantly different (p < 0.001) by ANOVA and Duncan's multiple range tests.

Experimental Result 2: Improvement of Wrinkles

1. Antioxidant Activity

1-1. Total Polyphenol Contents

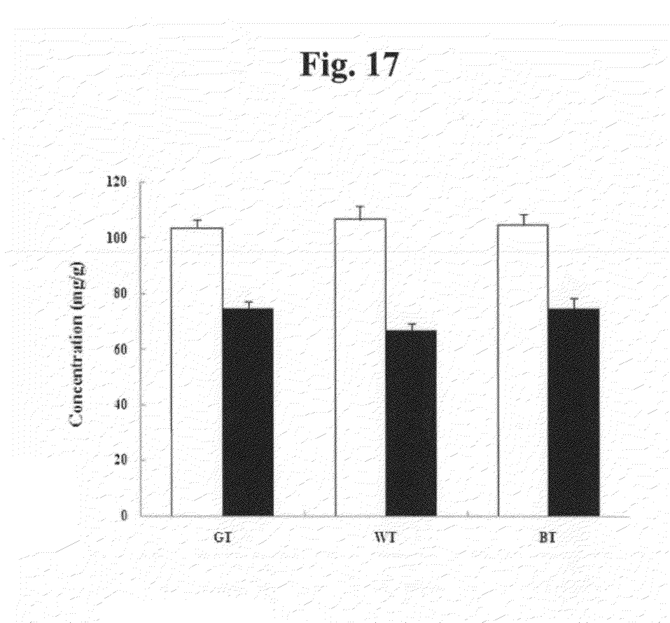

Total polyphenol contents of GT, WT and BT were 103.5, 106.7 and 104.7 mg/g, respectively (see FIG. 17).

1-2. Total Flavonoid Contents

Total polyphenol contents of GT, WT and BT were 74.7, 66.9 and 74.8 mg/g, respectively (see FIG. 17).

1-3. Electron Donating Ability

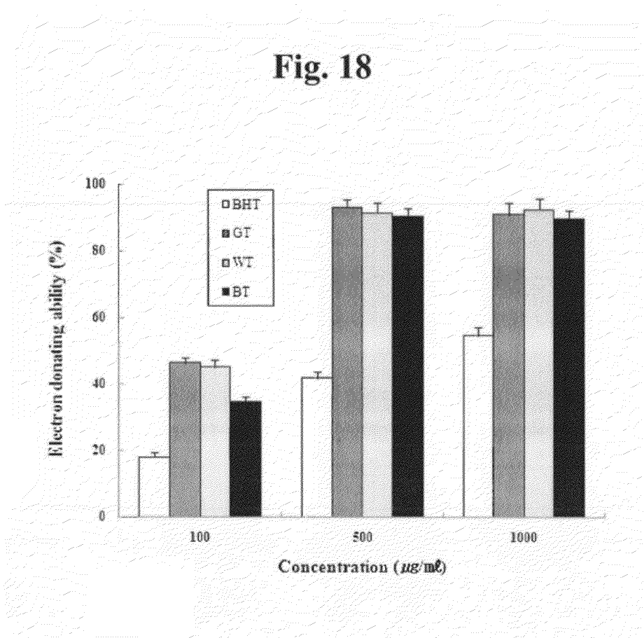

Electron donating ability of GT, WT and BT was 96, 91 and 90% at 1,000 μg/mL. The electron donating ability of BHT was 55% at the same concentration. Thus, the electron donating ability of GT, WT and BT was higher than that of BHT (see FIG. 18).

2. Change in Erythema Index

The result of skin erythema index measurement is shown in Table 6. All the test groups showed significantly lower skin erythema index during the test period as compared to the control group (p <0.001). On week 4, the skin erythema index of the groups RA, GT, WT and BT was 32, 41, 46 and 42% lower, respectively, as compared to the control group (p <0.001).

TABLE 6

| Weeks | | | Groups | | | |
|---|---|---|---|---|---|---|
| Weeks | N | C | RA 0.01% | GT 2% | WT 2% | BT 2% |
| 1 | 240.67 ± 30.67^{a} | 346.90 ± 14.48^{d} | 314.38 ± 17.33^{c} | 279.76 ± 11.72^{b} | 274.17 ± 5.87^{b} | 275.86 ± 5.64^{b} |
| 2 | 246.33 ± 23.30^{a} | 357.52 ± 14.46^{c} | 301.10 ± 16.60^{b} | 267.81 ± 12.00^{a} | 255.24 ± 8.46^{a} | 274.10 ± 11.51^{ab} |
| 3 | 261.71 ± 21.60^{a} | 377.43 ± 13.36^{c} | 294.43 ± 13.14^{b} | 260.93 ± 11.09³ | 236.75 ± 12.71^{a} | 261.31 ± 9.58^{a} |
| 4 | 285.50 ± 34.20^{b} | 413.43 ± 12.21^{c} | 282.64 ± 15.50^{b} | 245.50 ± 12.07^{a} | 223.64 ± 4.24^{a} | 238.86 ± 8.52^{a} |

Values are mean ± SD of 7 mice.
Unit: AU (Arbitrary Unit)
N: no UVB irradiation group
C: UVB irradiation + saline treatment group
RA 0.01%: UVB irradiation + 0.01% retinoic acid treatment group
GT 2%: UVB irradiation + 2% green tea treatment group
WT 2%: UVB irradiation + 2% white tea treatment group
BT 2%: UVB irradiation + 2% black tea treatment group
Values with different superscripts in the same row are significantly different (p < 0.001) by ANOVA and Duncan's multiple range tests.

3. Change in Water Content

The change in water content of the skin is shown in Table 7. All the test groups showed significantly higher water content during the test period as compared to the control group (p <0.001). On week 4, the water content of the groups RA, GT, WT and BT was 207, 196, 231 and 241% higher, respectively, as compared to the control group (p <0.001).

TABLE 7

| Weeks | | | Groups | | | |
|---|---|---|---|---|---|---|
| Weeks | N | C | RA 0.01% | GT 2% | WT 2% | BT 2% |
| 1 | 60.48 ± 9.53^{b} | 39.86 ± 1.59^{a} | 57.99 ± 7.31^{b} | 57.04 ± 10.01^{b} | 64.37 ± 2.25^{b} | 64.78 ± 2.39^{b} |
| 2 | 51.46 ± 9.66^{b} | 31.02 ± 1.75^{a} | 65.70 ± 5.75^{c} | 69.09 ± 9.84^{c} | 76.16 ± 2.66^{c} | 75.82 ± 1.31^{c} |
| 3 | 36.31 ± 7.25^{b} | 18.52 ± 3.09^{a} | 71.42 ± 5.82^{cd} | 69.61 ± 4.22^{c} | 78.79 ± 1.71^{de} | 80.90 ± 0.81^{e} |
| 4 | 46.06 ± 5.95^{b} | 28.90 ± 2.10^{a} | 88.59 ± 4.87^{cd} | 85.67 ± 5.38^{c} | 95.89 ± 2.42^{de} | 98.55 ± 5.98^{e} |

Values are mean ± SD of 7 mice.
Unit: AU (Arbitrary Unit)
N: no UVB irradiation group
C: UVB irradiation + saline treatment group
RA 0.01%: UVB irradiation + 0.01% retinoic acid treatment group
GT 2%: UVB irradiation + 2% green tea treatment group
WT 2%: UVB irradiation + 2% white tea treatment group
BT 2%: UVB irradiation + 2% black tea treatment group
Values with different superscripts in the same row are significantly different (p < 0.001) by ANOVA and Duncan's multiple range tests.

4. Change in TEWL

The change in TEWL of the skin is shown in Table 8. On week 2, all the test groups except for the group RA showed significantly decreased TEWL as compared to the control group (p <0.001). On week 4, the TEWL of the groups RA, GT, WT and BT was 58, 82, 87 and 86% lower, respectively, as compared to the control group (p <0.001).

TABLE 8

| Weeks | | | Groups | | | |
|---|---|---|---|---|---|---|
| Weeks | N | C | RA 0.01% | GT 2% | WT 2% | BT 2% |
| 1 | 8.29 ± 2.07^{a} | 19.16 ± 2.92^{bc} | 24.16 ± 4.49^{c}** | 14.67 ± 3.01^{b}** | 14.21 ± 2.03^{b}** | 13.81 ± 1.68^{b}** |
| 2 | 11.08 ± 2.37^{a} | 22.86 ± 2.71^{b} | 18.33 ± 4.85^{b}** | 11.66 ± 2.24^{a} | 10.79 ± 5.47^{a} | 9.84 ± 1.53^{a} |
| 3 | 11.90 ± 1.26^{b} | 24.79 ± 1.51^{d} | 18.76 ± 3.66^{c} | 9.86 ± 1.83^{ab} | 8.76 ± 1.45^{ab} | 7.74 ± 1.20^{a} |
| 4 | 15.77 ± 3.06^{b} | 40.27 ± 2.24^{c} | 16.90 ± 3.59^{b} | 7.39 ± 0.85^{a} | 5.34 ± 1.50^{a} | 5.53 ± 0.66^{a} |

Values are mean ± SD of 7 mice.
Unit: g/m²/h
N: no UVB irradiation group
C: UVB irradiation + saline treatment group
RA 0.01%: UVB irradiation + 0.01% retinoic acid treatment group
GT 2%: UVB irradiation + 2% green tea treatment group
WT 2%: UVB irradiation + 2% white tea treatment group
BT 2%: UVB irradiation + 2% black tea treatment group
Values with different superscripts in the same row are significantly different (p < 0.001) by ANOVA and Duncan's multiple range tests.
**p < 0.01 compared to the C group by ANOVA and Duncan's multiple range tests.

5. Morphological Change in Skin Tissue

5-1. Observation of Wrinkles

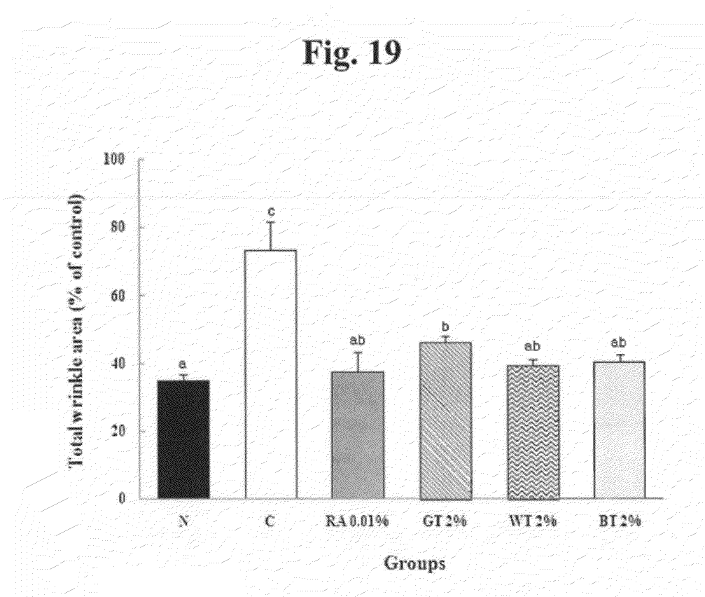

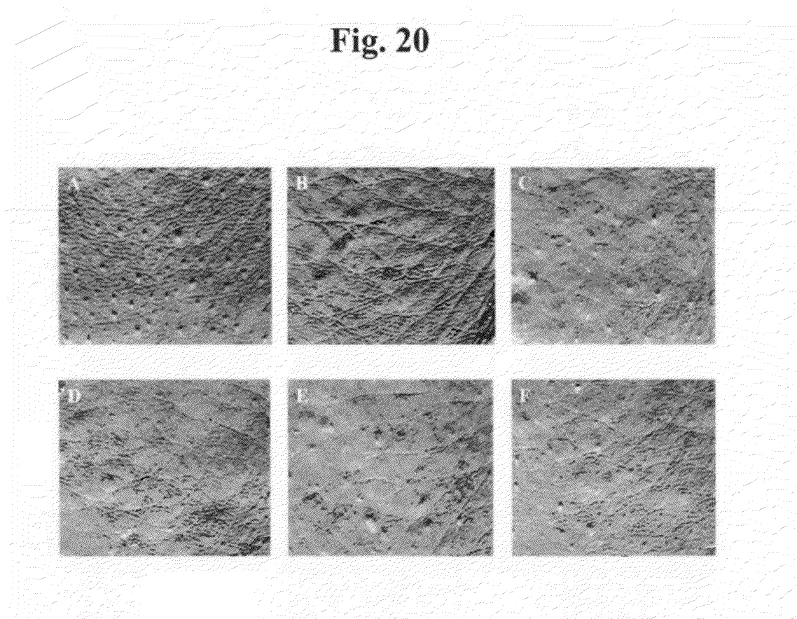

The result of skin wrinkle observation is shown in FIG. 19. When compared with the control group, the RA, GT, WT and BT administration groups showed improvement of wrinkles, with shallow wrinkles and narrow and thin crest pattern (see FIG. 20). To conclude, the control group showed 110% increased wrinkle area as compared to the normal group, and the groups RA, GT, WT and BT showed 49, 37, 46, and 45% decreased wrinkle area, respectively, as compared to the control group (p <0.001).

6. Histological Observation

6-1. Hematoxylin & Eosin Staining

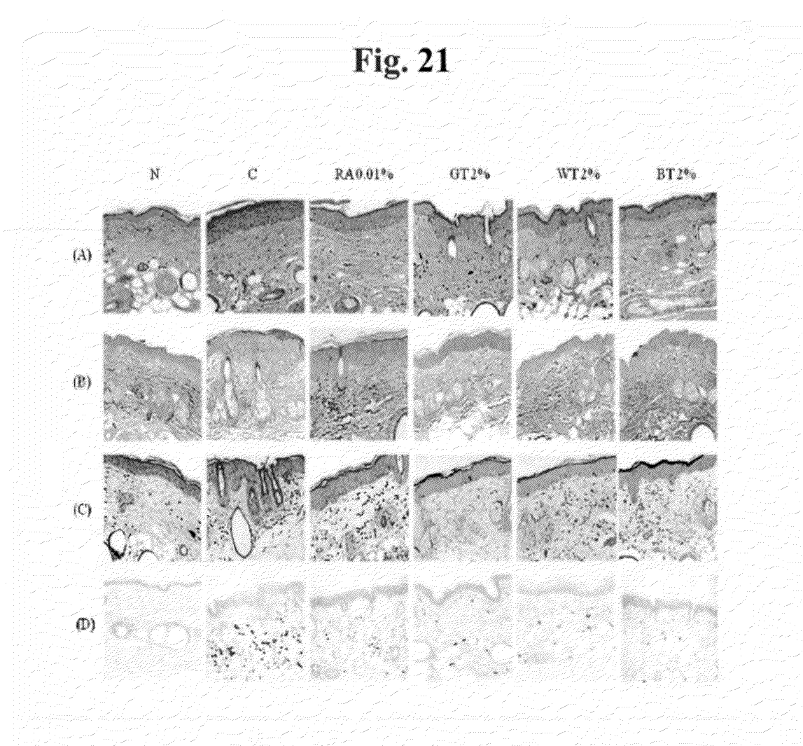

The result of skin tissue and inflammatory cell observation is shown in FIG. 21. The thickness of the epithelium and the skin of the control group was significantly increased, and a considerable number of inflammatory cells including lymphocytes, neutrophils and macrophages penetrated into the skin. In contrast, only a small number of inflammatory cells were observed in the skin of the groups RA, GT, WT and BT.

6-2. Masson's Trichrome Staining

The result of observing the quantity and shape of collagen fibers in the skin is shown in FIG. 21. The normal group showed regularly arranged, highly dense collagen fibers. In the skin of the control group, the collagen fibers were severely broken and the density was low. In the skin of the groups RA, GT, WT and BT, the collagen fibers were almost intact and showed regular arrangement.

6-3. Verhoeff Staining

The result of observing the quantity and shape of elastin fibers in the skin is shown in FIG. 21. The normal group showed regularly arranged elastin fibers. In contrast, the skin of the control group showed elastosis, with abnormal and entangled elastin fibers. The skin of the groups RA, GT, WT and BT showed less abnormality of elastin fibers as compared to the control group.

6-4. Toluidine Blue Staining

The result of observing the mast cell distribution pattern and degree of degranulation in the skin and the dermis is shown in FIG. 21. A large number of mast cells and severe degranulation were observed in the normal group. In contrast, a relatively small number of mast cells and very week degranulation were observed in the groups RA, GT, WT and BT.

7. Gene Expression in Skin Tissue

7-1. MMP-3

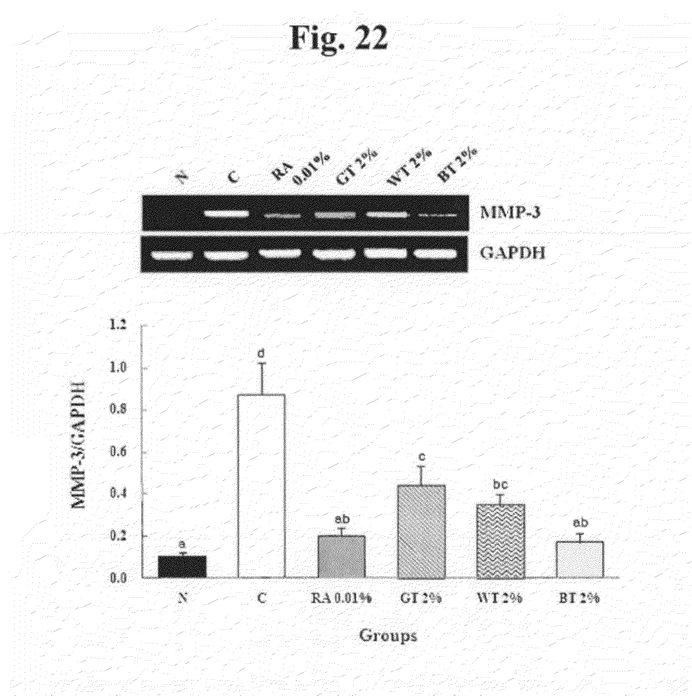

Expression of MMP-3 mRNA in the skin tissue was analyzed by RT-PCR (see FIG. 22). The expression level of MMP-3 mRNA in the groups RA, GT, WT and BT was lower as compared to the control group, with 77, 49, 60 and 80% (p <0.001).

7-2. Activity of MMP-2 and MMP-9 Proteins

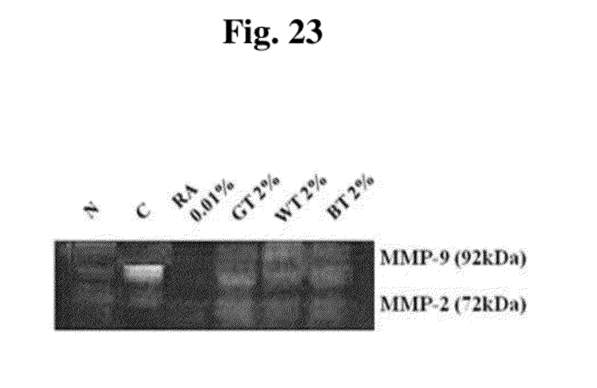

Activity of MMP-2 and MMP-9 proteins in the skin tissue was analyzed by zymography (see FIG. 23). The groups RA, GT, WT and BT showed remarkably lower activity as compared to the control group.

8. Change in Water and Feed Intake, Feed Efficiency and Body Weight

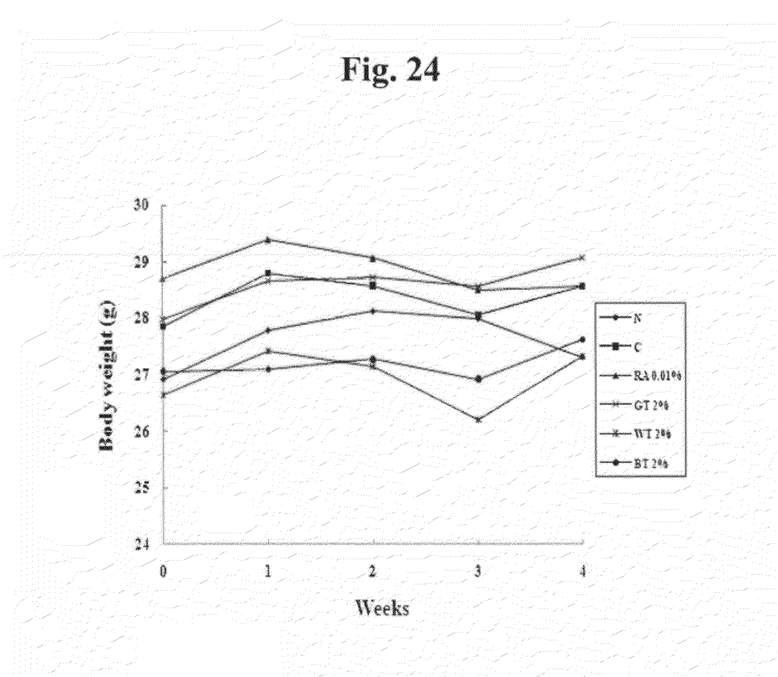

The result of measuring body weight, feed intake, water intake and feed efficiency is shown in Table 9 and FIG. 24. The group RA showed remarkably higher water intake as compared to the control group (p <0.05). In all test groups, feed intake, body weight increase and feed efficiency were not significantly different from the control group.

TABLE 9

| Items | N | C | RA 0.01% | GT 2% | WT 2% | BT 2% |
|---|---|---|---|---|---|---|
| Water | 6.25 ± 1.58^{a} | 5.36 ± 1.70^{a} | 8.57 ± 1.75^{b} | 6.43 ± 1.01^{ab} | 5.78 ± 1.18^{a} | 5.00 ± 0.88^{a} |
| intake | | | | | | |
| (Ml/day) | | | | | | |
| Food | 4.70 ± 0.91^{a} | 4.74 ± 1.27^{a} | 5.36 ± 1.06^{a} | 4.87 ± 0.97^{a} | 4.96 ± 1.30^{a} | 4.84 ± 1.05^{a} |
| intake | | | | | | |
| (g/day) | | | | | | |
| Body | −0.019 ± 0.11^{ab} | −0.026 ± 0.03^{ab} | −0.050 ± 0.09^{a} | 0.002 ± 0.06^{ab} | 0.03 ± 0.04^{ab} | 0.04 ± 0.52^{b} |
| weight | | | | | | |
| gain | | | | | | |
| (g/day) | | | | | | |
| Food | −0.40±2.40^{ab} | −0.54 ± 0.65^{ab} | −0.94 ± 1.71^{a} | 0.04 ± 1.20^{ab} | 0.60 ± 0.77^{ab} | 0.87 ± 1.06^{b} |
| efficiency | | | | | | |
| ratio¹⁾(%) | | | | | | |

Values are mean ± SD of 7 mice.
N: no UVB irradiation group
C: UVB irradiation + saline treatment group
RA 0.01%: UVB irradiation + 0.01% retinoic acid treatment group
GT 2%: UVB irradiation + 2% green tea treatment group
WT 2%: UVB irradiation + 2% white tea treatment group
BT 2%: UVB irradiation + 2% black tea treatment group
¹⁾Food efficiency ratio (%) = (Body weight gain/Food intake) × 100
Values with different superscripts in the same row are significantly different (p < 0.05) by ANOVA and Duncan's multiple range tests.

9. Change in Organ Weight

The result of measuring spleen and thymus weight is shown in Table 10. The absolute and relative weight of the spleen was not significantly different from the normal group. The weight of the control group and the groups RA and GT was significantly lower relative to the normal group (p <0.05).

TABLE 10

| Organs | N | C | RA 0.01% | GT 2% | WT 2% | BT 2% |
|---|---|---|---|---|---|---|
| Spleen | 0.130 ± 0.037^{1)a} | 0.103 ± 0.010^{a} | 0.136 ± 0.015^{a} | 0.141 ± 0.077^{a} | 0.113 ± 0.022^{a} | 0.241 ± 0.032^{a} |
| | 0.447 ± 0.107^{2)a} | 0.361 ± 0.036^{a} | 0.475 ± 0.053^{a} | 0.474 ± 0.211^{a} | 0.406 ± 0.081^{a} | 0.528 ± 0.071^{a} |
| Thymus | 0.023 ± 0.004^{1)b} | 0.020 ± 0.002^{ab} | 0.019 ± 0.005^{ab} | 0.016 ± 0.002^{a} | 0.021 ± 0.005^{b} | 0.020 ± 0.004^{ab} |
| | 0.087 ± 0.019^{2)c} | 0.070 ± 0.009^{ab} | 0.067 ± 0.01^{ab} | 0.056 ± 0.010^{a} | 0.077 ± 0.017^{bc} | 0.084 ± 0.011^{bc} |

Values are mean ± SD of 7 mice.
N: no UVB irradiation group
C: UVB irradiation + saline treatment group
RA 0.01%: UVB irradiation + 0.01% retinoic acid treatment group
GT 2%: UVB irradiation + 2% green tea treatment group
WT 2%: UVB irradiation + 2% white tea treatment group
BT 2%: UVB irradiation + 2% black tea treatment group
¹⁾Absolute weight: g
²⁾Relative weight: g/100 g body weight
Values with different superscripts in the same row are significantly different (p < 0.05) by ANOVA and Duncan's multiple range tests.

10. Hematological Change

Hematological analysis result is shown in Table 11. The groups GT, WT and BT showed smaller number of leukocytes, neutrophils and lymphocytes as compared to the control group (p <0.05). Erythrocyte concentration, hemoglobin concentration, hematocrit and platelet concentration of the control group were significantly lower than all other groups (p <0.05).

TABLE 11

| Items | N | C | RA 0.01% | GT 2% | WT 2% | BT 2% |
|---|---|---|---|---|---|---|
| WBC (k/μl) | 7.877 ± 0.14^{a} | 11.283 ± 0.41^{c} | 9.114 ± 0.55^{b} | 7.975 ± 0.10^{a} | 8.055 ± 0.09^{a} | 8.094 ± 0.16^{a} |
| Neutrophil (k/μl) | 1.736 ± 0.03^{a} | 2.530 ± 0.30^{d} | 2.340 ± 0.39^{cd} | 1.948 ± 0.04^{ab} | 1.805 ± 0.12^{a} | 2.174 ± 0.21^{bc} |
| Lymphocyte (k/μl) | 5.560 ± 0.39^{a} | 8.370 ± 0.57^{d} | 6.462 ± 0.34^{c} | 5.834 ± 0.15^{ab} | 6.157 ± 0.21^{bc} | 5.729 ± 0.13^{a} |
| Monocyte (k/μl) | 0.135 ± 0.11^{ab} | 0.083 ± 0.05^{a} | 0.204 ± 0.06^{b} | 0.150 ± 0.05^{ab} | 0.140 ± 0.05^{ab} | 0.143 ± 0.03^{ab} |
| Eosinophil (k/μl) | 0.034 ± 0.02^{a} | 0.040 ± 0.02^{a} | 0.050 ± 0.05^{a} | 0.062 ± 0.03^{a} | 0.052 ± 0.03^{a} | 0.026 ± 0.01^{a} |
| Basophil (k/μl) | 0.060 ± 0.11^{a} | 0.022 ± 0.03^{a} | 0.004 ± 0.04^{a} | 0.006 ± 0.04^{a} | 0.007 ± 0.05^{a} | 0.030 ± 0.08^{a} |
| RBC (M/μl) | 4.550 ± 0.80^{c} | 3.616 ± 0.12^{a} | 4.274 ± 0.17^{b} | 4.260 ± 0.05^{b} | 4.661 ± 0.14^{c} | 4.664 ± 0.11^{c} |
| Hb (g/dl) | 15.280 ± 0.56^{d} | 11.800 ± 0.46^{a} | 13.800 ± 0.18^{b} | 15.540 ± 0.37^{d} | 15.450 ± 0.52^{d} | 15.986 ± 0.53^{c} |
| HCT (%) | 45.84 ± 0.62^{cd} | 33.12 ± 0.96^{a} | 43.442 ± 0.87^{b} | 46.50 ± 0.26^{d} | 45.35 ± 0.75^{c} | 45.27 ± 0.67^{c} |
| Platelet (k/μl) | 544.80 ± 8.43^{c} | 438.27 ± 8.03^{a} | 517.80 ± 16.44^{b} | 518.0 ± 4.28^{b} | 556.7 ± 14.45^{c} | 557.0 ± 11.56^{c} |

Values are mean ± SD of 7 mice.
N: no UVB irradiation group,
C: UVB irradiation + saline treatment group
RA 0.01%: UVB irradiation + 0.01% retinoic acid treatment group
GT 2%: UVB irradiation + 2% green tea treatment group
WT 2%: UVB irradiation + 2% white tea treatment group
BT 2%: UVB irradiation + 2% black tea treatment group
Values with different superscripts in the same row are significantly different (p < 0.05) by ANOVA and Duncan's multiple range tests.

As described above, the present disclosure provides a composition for whitening skin or improving skin wrinkles comprising black tea extract as an active ingredient. In the cell experiment for Melan-a cells and the animal experiment using the brown guinea pig model with artificial tanning spots induced by UVB, the black tea extract of the present disclosure greatly inhibited melanin synthesis. Also, in the experiment on SKH-1 hairless mouse, it remarkably inhibited wrinkle formation. Accordingly, the composition of the present disclosure can be developed as a skin whitening agent, an anti-wrinkle agent or a medicine for treating diseases associated with melanin hyperpigmentation, e.g. liver spots, freckles, senile spots or solar lentigo.

While the present disclosure has been described with respect to the specific embodiments, it will be apparent to those skilled in the art that various changes and modifications may be made without departing from the spirit and scope of the disclosure as defined in the following claims.

### REFERENCES

Agar, N., and A. R. Young. 2005. Melanogenesis: a photoprotective response to DNA damage. Mutation Research 571, No. 1: 121-132.
Al-Azzawie, H. F., and S. A. Mohamed-Saiel. 2006. Hypoglycemic and antioxidant effect of oleuropein in alloxan-diabetic rabbits. Life Science 78: 1371-1377.
Ammar, R. B., W. Bhouri, M. B. Sghaier, J. Boubaker, I. Skandrani, A. Neffati, I. Bouhlel, S. Kilani, A.-M. Mariotte, L. Chekir-Ghedira, M-G Dijoux-Franca, and K. Ghedira. 2009. Antioxidant and free radical-scavenging properties of three flavonoids isolated from the leaves of Rhamnus alaternus L. (Rhamnaceae): a structure-activity relationship study. Food Chemistry 116: 258-264.
Ando, H., H. Kondoh, M. Ichihashi, and V. J. Hearing. 2007. Approaches to identify inhibitors of melanin biosynthesis via the quality control of tyrosinase. Investigative Dermatology 127, No. 4: 751-761.
Anna, G-M. 2008. Antioxidant potential and radical scavenging activity of different fermentation degree tea leaves extracts. International Journal of Tea Science 6: 15-28.
Balentine, D. 1992. Manufacturing and chemistry of tea. In Phenolic compounds in food and their effects on health. Edited by C.-T. Ho, T. Osawa, M.-T. Huang, and R. T. Rosen. Washington, D.C.: American Chemical Society. pp. 102-117.
Battaini, G., E. Monzani, L. Santagostini, and R. Pagliarin. 2000. Inhibition of the catecholase activity of biomimetic dinuclear copper complexes by kojic acid. Journal of Biological Inorganic Chemistry 5(2): 262-268.
Bennett, D., B. Cooper, and I. Hart. 1987. A line of non-tumorigenic mouse melanocytes, syngeneic with the B16 melanoma and requiring a tumour promoter for growth. International Journal of Cancer 39: 414-418.
Blois, M. S. 1958. Antioxidant determination by the use of a stable free radical. Nature 181: 1199-1200.
Bondent, V., W. Brand-Williams, and C. Bereset. 1997. Kinetic and mechanism of antioxidant activity using the DPPH free radical methods. Lebensmittel Wissenschaft and Technologie 30: 609-615.
Briganti, S., E. Camera, and M. Picardo. 2003. Chemical and instrumental approaches to treat hyperpigmentation. Pigment Cell Research 16: 101-110.
Chakraborty, A. K., Y. Funasaka, M. Komoto, and M. Ichihashi. 1998. Effect of arbutin on melanogenic proteins in human melanocytes. Pigment Cell Research 11: 206-211.
Choi, S. Y., S. Kim, J. S. Hwang, B. G. Lee, H. Kim, and S. Y. Kim. 2004. Benzylamide derivative compound attenuates the ultraviolet B-induced hyperpigmentation in the brownish guinea pig skin. Biochemical Pharmacology 67, No. 4: 707-715.
Clarys, P., K. Alewaeters, R. Lambrecht, and A. O. Bare. 2000. Skin color easurements: comparison between three instruments: the chromameter, the dermaspectrometer and the mexameter. Research and Technology 6: 230-236.
Cullen, M. K. 1998. Genetic epidermal syndromes: disorders characterized by lentigines. In the pigmentary system: physiology and pathophysiology. Edited by J. J. Norlund, R. E. Boissy, V. J. Hearing, R. A. King, and J. P. Ortonne. New York: Oxford University Press. pp. 760-766.
Davis, R., R. C. Massey, and D. J. Mcweeny. 1980. The catalisis of the N-nitrosation of secondary amines by nitrosophenols. Journal of Food Chemistry 6: 115-122.
Del Rio, D., A. J. Stewart, W. Mullen, J. Burns, M. E. J. Lean, and F. Brighenti, and A. Crozier. 2004. HPLC-MSn analysis of phenolic compounds and purine alkaloids in green and black teas. Journal of Agricultural and Food Chemistry 52: 2807-2815.
Draelos, Z. D. 2007. Skin lightening preparations and the hydroquinone controversy. Dermatologic Therapy 20: 308-313.
Finger, A., S., Kuhr, and U. H., Engelhardt. 1992. Chromatography of tea constituents. Journal of Chromatography 624: 293-315.
Folin, O., and W. Denis. 1912. On phosphotungstic-phosphomolybdic compounds as color reagents. Journal of Biochemistry and Chemistry 12: 239-249.
Graham, H. N. 1992. Green tea composition, consumption, and polyphenol chemistry. Preventive Medicine 21: 334-350.
Hearing, V. J. 2005. Biogenesis of pigment granules: a sensitive way to regulate melanocyte function, Journal of Dermatological Science 37: 3-14.
Hilal, Y., and U. Engelhardt. 2007. Characterization of white tea in comparison to green and, black tea. Journal fur Verbraucherschutz and Lebensmittelsicherheit 2: 414-421.
Hirobe, T. 1982. Origin of melanosome structures and cytochemical localization of tyrosinase activity in differentiating epidermal melanocytes of newborn mouse skin. Journal of Experimental Zoology 224: 355-363.
Hori, I., K. Nihei, and I. Kubo. 2004. Structural criteria for depigmenting mechanism of arbutin. Phytotherapy Research 18: 469-475.
Im, S., J. Kim, W. Y. On, and W. H. Kang. 2002. Increased expression of a-melanocyte-stimulating-hormone in the lesional skin of melasma. British Journal of Dermatology 146: 165-167.
Imokawa, G., M. Kawai, Y. Mishima, and I. Motegi. 1986. Differential analysis of experimental hypermelanosis induced by UVB, UVA, and allergic contact dermatitis using a brownish guinea pig model. Archives of Dermatology Research 278, No. 5: 352-362.
Kahkonen, M. P., A. I. Hopia, H. J. Vuorela, J.-P. Rauha, K. Pihlaja, T. S. Kujala, and M. Heinonen. 1999. Antioxidant activity of plant extracts containing phenolic compounds. Journal of Agricultural and Food Chemistry 47: 3954-3962.
Kang, S. K., J. S. Park, and K. R. Kim. 2007. A study on the facial skin erythema index and melanin index and the general factors. Journal of Korean Society of Cosmetology 13, No. 2: 507-513.
Kang, W. H., K. H. Yoon, E. S. Lee, J. Kim, K. B. Lee, H. Yim, S. Sohn, and S. Im. 2002. Melasma: histopathological characteristics in 56 Korean patients. British Journal of Dermatology 146: 228-237.
Karg, E, G. Odh, A. Wittbjer, E. Rosengren, and H. Rorsman. 1993. Hydrogen peroxide as inducer of elevated tyrosinase level in melanoma cells. Journal of Investigative Dermatology 100: 209s-213s.
Kikuchi, A., H. Shimizu, and T. Nishikawa. 1996. Expression and ultrastructural localization of HMB-45 antigen. British Journal of Dermatology 135: 400-405.
Khokhar, S., and S. G. M. Magnusdottir. 2002. Total phenol, catechin and caffeine contents of teas commonly consumed in the United Kingdom. Journal of Agricultural and Food Chemistry 50: 565-570.
Kim, D. S., S. H. Park, S. B. Kwon, K. S. Li, S. W. Youn, and K. C. Park. 2004. (−)-Epigallocathchin-3-gallate and hinokitol reduce melanin synthesis via decresed MITF production. Archives of Pharmacological Research 27, No. 3: 334-339.
Lim J. T. 1999. Treatment of melasma using kojic acid in a gel containing hydroquinone and glycolic acid. Dermatological Surgery 25, No. 4: 282-284.
Lo Scalzo, R. 2008. Organic acids influence on DPPH scavenging by ascorbic acid. Food Chemistry 107: 40-43.
Maeda, K., and M. Hatato. 2004. Involvement of photooxidation of melanogenic precursors in prolonged pigmentation induced by ultraviolet A. Journal of Investigative Dermatology 122: 503-509.
Maeda, K., and M. Fukuda. 1996. Arbutin: mechanism of its depigmenting action in human melanocyte culture. Journal of Pharmacology and Experimental Therapy 276: 765-769.
Mallick, S., S. K. Singh, C. Sarkar, B. Saha, and R. Bhadra. 2005. Human placental lipid induces melanogenesis by increasing the expression of tyrosinase and its related proteins in vitro. Pigment Cell Research 18: 25-33.
Manzocco, L., M. Anese, and M. C. Nicoli. 1998. Antioxidant properties of extracts as affected by processing. Lebensmittel Wissenchard and Technology 31; 694-698.
Masson, P. 1928. Carcinoids (Argentaffin-cell tumors) and nerve hyperplasia of the appendicular mucosa. American Journal of Pathology 4, No. 3: 181-211.
Menter, J. M., A. A. Etemadi, W. Chapman, T. D. Hollins, and I. Willis. 1993. In vivo depigmentation by hydroxybenzene derivatives. Melanoma Research 6: 443-449.
Mizukami, Y., Y. Sawai, and Y. Yamaguchi. 2007. Simultaneous analysis of catechins, gallic acid, strictinin, and purine alkaloids in green tea by using catechol as an internal standard. Journal of Agricultural and Food Chemistry 55: 4957-4964.
No, J. K., D. Y. Soung, Y. J. Kim, K. H. Shim, Y. S. Jun, S. H. Rhee, T, Yokozawa, and H. Y. Chung. 1999. Inhibition of tyrosinase by green tea components. Life Science 65: PL241-PL246.
Ortonne, J. P., and J. J. Nordlund. Mechanisms that cause abnormal skin color. 1998. In the pigmentary system: physiology and pathophysiology. Edited by J. J. Norlund, R. E. Boissy, V. J. Hearing, R. A. King, and J. P. Ortonne. New York: Oxford University Press. pp. 489-502.
Ortonne, J. P., and T. Passeron. 2005. Melanin pigmentary disorders: treatment update. Dermatologic Clinics 23(2): 209-226.
Passi, S, and M. Nazzaro-Porro. 1981. Molecular basis of substrate and inhibitory specificity of tyrosinase: phenolic compounds. British Journal of Dermatology 104: 659-665.
Pathak, M. A., and T. B. Fitzpatrick. 1993. Preventive treatment of sunburn, dermatoheliosis, and skin cancer with sun protective agents. New York: McGraw-Hill.
Serafini, M., A. Ghiselli, and A. Ferro-Luzzi. 1996. In vivo antioxidant effect of green tea and black tea in man. European Journal of Clinical Nutrition 50: 28-32.
Seyoum, A., K. Asres, and F. K. E1-Fiky. 2006. Structure-radical scavenging activity relationships of flavonoids. Phytochemistry 67: 2058-2070.
Sheffield, M. V., H. Yee, C. C. Dorvault, K. N. Weilbaecher, I. A. Eltoum, and G. P. Siegal. 2002. Comparison of five antibodies as markers in the diagnosis of melanoma in cytologic preparations. American Journal of Clinical Pathology 118, No. 6: 930-936.
Sun, C. L., J. M. Yuan, W. P. Koh, and M. C. Yu. 2006. Green tea, black tea and breast cancer risk: a meta-analysis of epidemiological studies. Carcinogenesis 27, No. 7: 1310-1315.
Tobiishi, M., A. Haratake, H. Kaminaga, M. Nakahara, A. Komiya, H. Koishikawa, H. Uchiwa, Y. Kawa, and M. Mizoguchi. 2004. Pigmentation in intrinsically aged skin of A1 guinea pigs. Pigment Cell Research 17, No. 6: 651-658.
Tripathi, R. K., V. J. Hearing, K. Urabe, P. Aroca, and R. A. Spritz. 1992. Mutational mapping of the catalytic activities of human tyrosinase. Journal of Biological Chemistry 267: 23707-23712.
Urabe K, J. Nakayama, and Y. Hori. 1998. Mixed epidermal and dermal hypermelanoses. In the pigmentary system: physiology and pathophysiology. Edited by J. J. Norlund, R. E. Boissy, V. J. Hearing, R. A. King, and J. P. Ortonne. New York: Oxford University Press. pp. 909-911.
Yoshida, M., S. Hirotsu, M. Nakahara, H. Uchiwa, and Y. Tomita. 2002. Histamine is involved in ultraviolet B-induced pigmentation of guinea pig skin. Journal of Investigative Dermatology 118: 255-260.
Wang, H., G. J. Provan, and K. Helliwell. 2000. Tea flavonoids, their functions, utilization and analysis. Trends in Food Science and Technology 11: 152-160.
Wang, Y., and C.-T. Ho. 2009. Polyphenolic chemistry of tea and coffee: A century of progress. Journal of Agricultural and Food Chemistry 57: 8109-8114.

What is claimed is:

1. A method of skin treatment, which comprises topically applying a topical composition comprising black tea extract on one or more areas of the skin of a person, thereby achieving reduction of pigmentation spots or degree of wrinkles.

2. The method according to claim 1, wherein said black tea extract is extracted using an extraction solvent selected from the group consisting of water, absolute or aqueous lower alcohol containing 1-4 carbons, acetone, ethyl acetate, butyl acetate, dichloromethane (CH₂Cl₂), chloroform, hexane and 1,3-butylene glycol.

3. The method according to claim 1, wherein said black tea extract is included in an amount of 0.0001-10 wt % based on the total weight of the composition.

4. The method according to claim 1, wherein said topical composition is a formulation in the form selected from the group consisting of solution, suspension, emulsion, paste, gel, cream, lotion, powder, soap, surfactant-containing cleanser, oil, powder foundation, emulsion foundation, wax foundation and spray.


SEQUENCE LISTING (12 sequences)

SEQ ID NO: 1 - DNA, 20 bp, Artificial Sequence
  PCR forward primer for Tyrosinase
catttttgatttgagtgtct

SEQ ID NO: 2 - DNA, 20 bp, Artificial Sequence
  PCR reverse primer for Tyrosinase
tgtggtagtcgtctttgtcc

SEQ ID NO: 3 - DNA, 21 bp, Artificial Sequence
  PCR forward primer for TRP-1
gctgcaggagccttctttctc

SEQ ID NO: 4 - DNA, 21 bp, Artificial Sequence
  PCR reverse primer for TRP-1
aagacgctgcactgctggtct

SEQ ID NO: 5 - DNA, 21 bp, Artificial Sequence
  PCR forward primer for TRP-2
ggatgaccgtgagcaatggcc

SEQ ID NO: 6 - DNA, 21 bp, Artificial Sequence
  PCR reverse primer for TRP-2
cggttgtgaccaatgggtgcc

SEQ ID NO: 7 - DNA, 20 bp, Artificial Sequence
  PCR forward primer for beta-actin
accgtgaaaagatgacccag

SEQ ID NO: 8 - DNA, 20 bp, Artificial Sequence
  PCR reverse primer for beta-actin
tacggatgtcaacgtcacac

SEQ ID NO: 9 - DNA, 21 bp, Artificial Sequence
  PCR forward primer for MMP-3
tagcaggttatcctaaaagca

SEQ ID NO: 10 - DNA, 19 bp, Artificial Sequence
  PCR reverse primer for MMP-3
ccagctattgctcttcaat

SEQ ID NO: 11 - DNA, 20 bp, Artificial Sequence
  PCR forward primer for GAPDH
cccactaacatcaaatgggg

SEQ ID NO: 12 - DNA, 20 bp, Artificial Sequence
  PCR reverse primer for GAPDH
acacattgggggtaggaaca